(19) United States
(12) Patent Application Publication
(10) Pub. No.: US 2025/0262349 A1
(43) Pub. Date: Aug. 21, 2025

(54) HYBRID BIOINK BIOMATERIAL

(71) Applicant: Board of Trustees of Michigan State University, East Lansing, MI (US)

(72) Inventors: Nureddin A. SHAMMAKHI, East Lansing, MI (US); Xanthippi CHATZISTAVROU, Okemos, MI (US)

(73) Assignee: Board of Trustees of Michigan State University, East Lansing, MI (US)

(21) Appl. No.: 18/566,824

(22) PCT Filed: Jun. 3, 2022
(86) PCT No.: PCT/US2022/032085

§ 371 Date: Dec. 4, 2023

Related U.S. Application Data
(60) Provisional application No. 63/196,743, filed on Jun. 4, 2021.

(51) Int. Cl. A61L 27/22; A61L 27/38; A61L 27/44; A61L 27/52; A61L 27/54; B33Y 70/00

(52) U.S. Cl. CPC A61L 27/222; A61L 27/3804; A61L 27/446; A61L 27/52; A61L 27/54; B33Y 70/00; A61L 2300/404; A61L 2400/12; A61L 2430/02

(57) ABSTRACT

A biomaterial is provided. The biomaterial includes a methacrylated polymer-silver-containing bioactive glass (methacrylated polymer-AgBaG) in which the AgBaG is chemically coupled to the methacrylated polymer. The methacrylated polymer may include gelatin methacryloyl (GelMA). Methods of making and using the biomaterial are also provided.

### CROSS-REFERENCE TO RELATED APPLICATIONS

This application is a 371 U.S. National Phase of International Application No. PCT/US2022/032085, filed on Jun. 3, 2022, and also claims the benefit of U.S. Provisional Application No. 63/196,743, filed on Jun. 4, 2021. The entire disclosures of the above applications are incorporated herein by reference.

### FIELD

The present disclosure relates to a hybrid bioink biomaterial including a bioactive glass (BaG) moiety chemically bonded to gelatin methacryloyl (GelMA).

### BACKGROUND

This section provides background information related to the present disclosure which is not necessarily prior art.

Various bioinks have been developed to three-dimensionally (3D) bioprint various constructs. For the 3D bioprinting of hard tissues, such as bone, osteoconductive and osteopromotive elements are required. Particles of hydroxyapatite (HAp), silica, or BaG have been added to bioinks for this purpose. Unfortunately, these particles coexist with the matrix material mostly in physical association, and they may lead to injury of cells in the bioink during the printing process of extrusion, the most commonly used method for 3D bioprinting. In addition, physical mixing of two phases in the bioink cannot always ensure the homogeneity of the end product, nor can allow for the control of the composition to generate, for example, gradients. Therefore, new bioinks that provide homogenous end products and have improved cellular viability after extrusion-based 3D bioprinting are desired.

### SUMMARY

This section provides a general summary of the disclosure, and is not a comprehensive disclosure of its full scope or all of its features.

In various aspects, the current technology provides a biomaterial including gelatin methacryloyl (GelMA) and a silver (Ag)-containing BaG (AgBaG) moiety, wherein the AgBaG moiety is chemically attached to the GelMA to define GelMA-AgBaG.

In one aspect, the AgBaG moiety is chemically attached to the GelMA by way of a covalent bond.

In one aspect, the AgBaG moiety is chemically attached to the GelMA by way of a linker.

In one aspect, the linker is (3-glycidyloxypropyl) trimethoxysilane (GPTMS).

In one aspect, the AgBaG moiety includes oxides of silicon, calcium, and phosphorus.

In one aspect, the AgBaG moiety further includes at least one oxide of aluminum, sodium, or potassium.

In one aspect, the AgBaG moiety further includes silver oxide (Ag₂O), silver ions (Ag⁺), or a combination thereof.

In one aspect, the GelMA-AgBaG is dissolved in an aqueous solvent.

In one aspect, the biomaterial further includes a photoinitiator.

In one aspect, the biomaterial further includes a plurality of cells.

In one aspect, the biomaterial further includes a plurality of cell-derived products

In one aspect, the plurality of cell-derived products includes extracellular vesicles.

In one aspect, the biomaterial further includes an additive selected from the group consisting of growth factors, antimicrobial agents, active pharmaceutical ingredients, polynucleotides, and combinations thereof.

In one aspect, the biomaterial further includes an adjunct agent selected from the group consisting of preservatives, visualization markers, imaging agents, monitoring agents, agents for increasing efficiency, agents for increasing cytocompatibility, agents for increasing biocompatibility, agents for modulating an immune response, agents for improving function of the biomaterial, agents for providing an additional function to the biomaterial, and combinations thereof.

In various aspects, the current technology provides a biomaterial including a methacrylated polymer and a silver (Ag)-containing BaG (AgBaG) moiety, wherein the AgBaG moiety is chemically attached to the methacrylated polymer to define methacrylated polymer-AgBaG.

In various aspects, the current technology also provides a construct including the biomaterial, wherein the biomaterial is crosslinked.

In one aspect, the construct is configured as a scaffold.

In one aspect, the construct includes at least one type of living cell (e.g., mammalian, bacteria, etc.) or cellular product (e.g., extracellular matrix (ECM) or ECM derivative) embedded within the crosslinked biomaterial.

In various aspects, the current technology further provides a method of treating a disorder or augmenting a structure or function of a tissue in a subject in need thereof, the method including disposing the construct on a location of the disorder or the tissue in the subject.

In one aspect, the disorder is a bone defect, and the method includes disposing the construct in a site of the bone defect.

In one aspect, the construct is a patient-specific construct having a 3D geometry that is a negative of the bone defect.

In one aspect, the disorder is a hemorrhage, and the method includes disposing the construct at a source of the hemorrhage.

In one aspect, the disorder is an aneurysm, and the method includes disposing the construct at the site of the aneurysm.

In one aspect, the disorder is a skin defect, and the method includes disposing the construct at the site of the skin defect, wherein the skin defect is a wound, burn, pressure sore, or excision.

In one aspect, the disorder is inflamed tissue associated with osteomyelitis, periimplantitis, periodontitis, or combinations thereof, and the method includes disposing the construct at the site of the inflamed tissue.

In various aspects, the current technology additionally provides a method of making the construct, the method including 3D printing the construct from the biomaterial and crosslinking the biomaterial to form the construct.

In various aspects, the current technology also provides a method of treating a defect or augmenting a structure or function of a tissue in a subject in need thereof, the method including applying the biomaterial to a target site associated with the defect or the tissue and crosslinking the biomaterial in situ to form a 3D construct including the GelMA-AgBaG at the target site.

In various aspects, the current technology further provides a method of making a biomaterial, the method including combining a first solution including GelMA, a linker, and a second solution including glass-ceramic precursors and Ag to form a biomaterial precursor solution and incubating the biomaterial precursor solution at greater than or equal to about 25° C. to less than or equal to about 100° C. for a time sufficient to form the biomaterial including GelMA-AgBaG, wherein the AgBaG is chemically coupled to the GelMA by way of the linker.

In one aspect, the first solution includes the GelMA dissolved in phosphate buffered saline (PBS).

In one aspect, the second solution includes the glass-ceramic precursors and Ag dissolved in an aqueous solvent, wherein the glass-ceramic precursors include a silicon oxide (SiO₂) precursor, a calcium oxide (CaO) precursor, a phosphorus pentoxide (P₂O₅) precursor, and optionally at least one of an aluminum oxide (Al₂O₃) precursor, a sodium oxide (Na₂O) precursor, or a potassium oxide (K₂O) precursor.

In one aspect, the incubating is performed for greater than or equal to about 1 hour to less than or equal to about 24 hours.

In one aspect, the linker is GPTMS.

In one aspect, the method further includes combining a photoinitiator with the first solution, the linker, and the second solution.

In one aspect, the GelMA is prepared by incubating gelatin type A and methacrylic acid (MAA) in a carbonate-bicarbonate (CB) buffer with a pH of 7-9 or PBS with a pH of 7-9 for greater than or equal to about 0.5 hours to less than or equal to about 12 hours to form a GelMA solution, filtering the GelMA solution to remove impurities, and dialyzing the GelMA solution against water.

In one aspect, the GelMA is prepared by dissolving gelatin type A in dimethyl sulfoxide (DMSO) to form a gelatin solution, adding MAA to the gelatin solution over a time period of greater than or equal to about 0.5 hours to less than or equal to about 6 hours to form the GelMA in the gelatin solution, precipitating the GelMA by adding toluene to the gelatin solution at about 2 times to 5 times the volume of the DMSO in the gelatin solution, removing the DMSO and the toluene to isolate the GelMA, and washing the GelMA with water.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1I are illustrations showing various classes of hybrids; FIG. 1A depicts Class I hybrids; FIG. 1B is a detail view of FIG. 1A; FIG. 1C depicts Class II hybrids; FIG. 1D is a detail view of FIG. 1C; FIG. 1E depicts Class III hybrids; FIG. 1F is a detail view of FIG. 1F; FIG. 1G depicts Class IV hybrids; FIG. 1H is a detail view of FIG. 1G; and FIG. 1I depicts Class V hybrids.

FIG. 2 shows a first reaction scheme for generating GelMA in accordance with various aspects of the current technology.

FIG. 3 shows proton nuclear magnetic resonance (¹H-NMR) spectra of GelMA made through the reaction scheme of FIG. 2.

FIG. 4 shows Fourier-transform infrared spectroscopy (FTIR) spectra of GelMA made through the reaction scheme of FIG. 2.

FIG. 5 shows a second reaction scheme for generating GelMA in accordance with various aspects of the current technology.

FIG. 6 shows ¹H-NMR spectra of GelMA made through the reaction scheme of FIG. 5.

FIG. 7 shows FTIR spectra of GelMA made through the reaction scheme of FIG. 5.

FIGS. 8A-8D show a reaction scheme for making GelMA-AgBaG in accordance with various aspects of the current technology.

FIG. 9 is a graph showing storage modulus versus temperature of GelMA-AgBaG made in accordance with various aspects of the current technology.

FIG. 10 is a graph showing apparent viscosity versus shear rate of GelMA-AgBaG made in accordance with various aspects of the current technology.

FIG. 11 shows a first set of FTIR spectra of GelMA-AgBaG made in accordance with various aspects of the current technology.

FIG. 12 shows a second set of FTIR spectra of GelMA-AgBaG made in accordance with various aspects of the current technology.

FIG. 13 shows scanning electron microscopy/energy dispersive X-ray spectroscopy (SEM/EDS) images of GelMA-AgBaG made in accordance with various aspects of the current technology.

FIG. 14 is a plot showing the printability of GelMA-AgBaG made in accordance with various aspects of the current technology.

FIG. 15 is a table showing theoretical shear rate and viscosity for GelMA-AgBaG hybrid hydrogels calculated using the shear thinning coefficients (i.e., n and K), hypothetical pressures, and hypothetical needle geometry.

FIG. 16 is a graph showing the antibacterial activity of GelMA-AgBaG made in accordance with various aspects of the current technology.

FIG. 17 is a graph showing mass loss of GelMA, GelMA-AgBaG blend, and GelMA-Ag-BaG hybrid made in accordance with various aspects of the current technology over a period of 7 days.

FIG. 18 is a graph showing mass loss of GelMA, GelMA-AgBaG blend, and GelMA-Ag-BaG hybrid made in accordance with various aspects of the current technology over a period of 30 days.

FIG. 19 is a graph showing swelling ratios of GelMA, GelMA-AgBaG blend, and GelMA-Ag-BaG hybrid made in accordance with various aspects of the current technology.

FIGS. 20A-20D relate to a molecular schematic of the synthesis used to produce GAB along with the process applied to deliver GAB hydrogels in accordance with various aspects of the current technology.

FIG. 21A depicts ¹H-NMR spectra of gelatin (as-received), GelMA, and GAB in accordance with various aspects of the present technology, with optical image inserts to show the status of the hydrogels post-synthesis, arrows to denote the lysine methylene signals used to determine the degree of substitution, and a zoomed in spectra within this range shown as an insert for clarity.

FIG. 21B depicts FTIR spectra of the gelatin (as-received), GelMA, GAB, the Ag-BaG solution used during the GAB synthesis, methanol (MeOH), the coupling agent (3-Glycidyloxypropyl) trimethoxysilane (GPTMS), MAA used for the methacrylation of the as-received gelatin, and dimethyl sulfoxide (DMSO) used as the solvent for the synthesis of GelMA and GAB.

FIG. 22 depicts SEM images of (a, b) GelMA in addition to the respective EDS X-ray mapping, where (c) C, (d) N, (e) O, and (f) Cl are all found to be homogenously distributed down to the micron level. Additionally, SEM images of (g, h) GAB and corresponding EDS X-Ray mapping showing that (i) C, (j) N, (k) O, (l) Cl, (m) Si, (n) Ca, (o) Al, (p) Ag, and (q) Na are all homogeneously distributed down to the micron-level in accordance with various aspects of the present disclosure.

FIG. 23A is a graph depicting the swelling ratio of GelMA, the GelMA-Ag-BaG nanocomposite, and GAB in accordance with various aspects of the present disclosure.

FIG. 23B is a graph depicting the pH evolution of GelMA, the GelMA-Ag-BaG nanocomposite, and GAB after 1, 3, 5, and 7d of immersion in PBS in accordance with various aspects of the present disclosure.

FIG. 23C is a graph illustrating the corresponding mass loss for each time point of FIG. 23B in accordance with various aspects of the present disclosure.

FIG. 24A is a graph illustrating storage (G′) and loss (G″) modulus for GAB as a function of temperature ranging from 25° C. to 40° C. at 1 Hz of oscillation in accordance with various aspects of the present disclosure

FIG. 24B is a graph illustrating the apparent viscosity of the GAB as a function of the shear rate showing GAB exhibits shear thinning behavior in accordance with various aspects of the present disclosure.

FIG. 24C is a photograph depicting single mesh layer of GAB 3D printed in accordance with various aspects of the present disclosure.

FIGS. 25A-25D relate to a schematic depicting synthesis of GelMA-Ag-BaG hybrid material in accordance with various aspects of the present disclosure.

FIG. 26 depicts SEM/EDS images show elemental analysis that demonstrates the homogeneity of elements in GAB in accordance with various aspects of the present disclosure.

FIG. 27 is an SEM image showing the porous microstructure of printed GAB in accordance with various aspects of the present disclosure.

FIG. 28A is a graph illustrating swelling of GelMA, GelMA-Ag-BaG, and GAB after immersion in water at a concentration of 10% (w/v) in accordance with various aspects of the present disclosure.

FIG. 28B is a graph illustrating mass loss over time for GelMA, GelMA-Ag-BaG, and GAB, with GAB showing the least mass loss over 7 days of immersion in accordance with various aspects of the present disclosure.

FIGS. 29A-29B relate to rheological characteristics of GAB material, which define its capability to be used in 3D printing in accordance with various aspects of the present disclosure; FIG. 29A is a graph illustrating apparent viscosity as a function of shear rate; FIG. 29B is a graph illustrating storage/loss modulus as a function of temperature.

FIG. 30 is an optical image showing printed GAB (15 psi, 27G, 4 mm/s) in accordance with various aspects of the present disclosure.

FIG. 31 is a graph illustrating Inhibition of bacterial growth (*) p<0.05 comparing samples within the same time point, (#) p<0.05 comparing the same sample at different time points in accordance with various aspects of the present disclosure.

FIGS. 32A-32F are optical images illustrating the addition of 3T3 fibroblasts to a GAB hydrogel, crosslinked, and incubated in accordance with various aspects of the present disclosure; FIG. 32A shows proliferation after 24 hours; FIG. 32B shows proliferation after 48 hours; FIG. 32C shows proliferation after 48 hours at a higher zoom than FIG. 32B; FIG. 32D shows proliferation after 72 hours; FIG. 32E shows proliferation after 72 hours at a higher zoom than FIG. 32D; and FIG. 32F shows proliferation after 72 hours at a higher zoom than FIG. 32E.

FIGS. 33A-33B are graphs illustrating metabolic activity of MSCs and fibroblasts under hypoxic conditions (of 3T3 and MSC) in extruded 3D GelMA, GAB and blend constructs. (*) p<0.05 comparing samples within the same time point and (#) p<0.05 comparing the same sample at the two different time points in accordance with various aspects of the present disclosure. The graphs depict an increase in cell viability for the extruded samples of GelMA and GAB after 3 days of culture, with cell viability decreasing after 3 days of culture in the extruded blend constructs.

FIGS. 34A-34B are graphs illustrating cell viability on as compared to control (2D culture) under hypoxic conditions (of 3T3 and MSC) for cultures up to 3 days in GAB samples without and with 0.5% wt. CPO incorporation in accordance with various aspects of the present disclosure.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

The current technology provides a hybrid methacrylated polymer-AgBaG-based biomaterial that is useful, for example, as a bioink for 3D bioprinting. The methacrylate polymer may include GelMA, hyaluronan, 2-hydroxyethyl methacrylate (HEMA), 3-(trimethoxysilyl) propyl methacrylate (TMSPMA), ethylene glycol dimethacrylate (EGDMA), poly(MMA-co-TMSPMA), or a combination thereof, by way of example.

In certain aspects, the methacrylated polymer includes GelMA. A first component, GelMA, has gelatin that provides tripeptide arginyl glycyl aspartic acid (RGD) motifs for facilitating cell attachment, migration, and proliferation and methacryloyl for enabling crosslinking following extrusion. A second component is AgBaG, which provides an anti-infective effect (e.g., antimicrobial activity) through both the Ag and the BaG and osteogenic and angiogenic effects through the BaG. The GelMA and the AgBaG are chemically associated, e.g., covalently attached. This hybrid biomaterial provides an improved cell viability relative to 3D bioprinted constructs produced by extrusion-based 3D bioprinting using, for example, physically-associated (not chemically-associated) or weakly-linked materials. Moreover, the hybrid biomaterial has a high degree of homogeneity that facilitates controlled degrees of building gradients in the bioink when desired and may be used for the engineering of interface tissues, such as osteochondral tissues, muscle-tendon, and tendon-bone or muscle-bone, as non-limiting examples. In this case, the hybrid material disclosed in this invention may be combined with other materials that have different properties suitable for the regeneration of type of tissue or the other or controlling gradients in the structure of one tissue itself. For example, GelMA may combined or substituted with materials such as methacrylated hyaluronic acid (HaMA).

The homogeneity facilitates the use of additives that are not affected by the presence of a second additive physically mixed with them. These include other particulate phases, biomolecules, such as growth factors or drugs, or polynucleotides, as non-limiting examples. When the hybrid biomaterial includes cells, the resulting construct can be used as a model to study function or disease, to develop and test drugs and other agents, or therapeutic modalities such as radiotherapy and phototherapy alone or in combination with other approaches/agents, and/or for regenerative and reparative/diagnostic or theranostic applications.

The hybrid biomaterial is biocompatible and useful in a variety of applications, including for in situ hemostasis to treat a hemorrhage, whether applied as injectable material, printed constructs, or delivered by other means to the site of bleeding. The biomaterial can also be used in the prophylactic treatment of aneurysms or to prevent or decrease their likelihood of rupture and catastrophic bleeding and in the treatment of wounds (e.g., acute or chronic) and tissues that have a high risk of infection, such as burns or traumatic wounds or wounds in immunocompromised patients. Given the antimicrobial (e.g., antibacterial) nature of the GelMA-AgBaG, the hybrid biomaterial is also useful to treat infections, such as osteomyelitis, periimplantitis, and periodontitis, as non-limiting examples. The hybrid biomaterial can be used alone or with other materials and/or devices.

Accordingly, the current technology provides a biomaterial comprising GelMA and a AgBaG moiety, wherein the AgBaG moiety is chemically attached to the GelMA to define GelMA-AgBaG. Because the AgBaG is chemically attached to the GelMA, the GelMA-AgBaG can be referred to as a hybrid biomaterial. By “chemically attached,” it is meant that the AgBaG moiety is covalently bonded to the GelMA, as opposed to a physical or non-covalent association. In some aspects, the AgBaG moiety is chemically attached to the GelMA by way of a linker, such as (3-Glycidyloxypropyl) trimethoxysilane (GPTMS), as a non-limiting example.

The AgBaG moiety comprises SiO₂, CaO, P₂O₅, and optionally at least one of Al₂O₃, Na₂O, or K₂O. The AgBaG moiety also comprises Ag₂O, Ag⁺, or a combination thereof. However, in alternative aspects, the biomaterial can include an Ag-free BaG, such as, GelMA-BaG. Therefore, it is understood that GelMA-AgBaG as recited herein can alternatively be GelMA-BaG, unless specifically stated otherwise.

In some aspects, the biomaterial comprises the GelMA-AgBaG dissolved in an aqueous solvent, such as water or PBS. A photoinitiator can also be included in the biomaterial to facilitate crosslinking and gelation of the GelMA component of the GelMA-AgBaG. Non-limiting examples of suitable photoinitiators include 2,2-Azobis (2-methyl-N-(2-hydroxyethyl) propionamide) (VA-086), lithium phenyl-2,4,6-trimethylbenzoylphosphinate (LAP), 2-hydroxy-1-[4-hydroxyethoxy)phenyl]-2-methyl-1-propanone (Irgacure 2959), and combinations thereof. The photoinitiator is included in the biomaterial at a first concentration of greater than or equal to about 1% (w/v) (e.g., greater than or equal to about 1.5% (w/v), greater than or equal to about 2% (w/v), greater than or equal to about 2.5% (w/v), greater than or equal to about 3% (w/v), greater than or equal to about 3.5% (w/v), greater than or equal to about 4% (w/v), greater than or equal to about 4.5% (w/v), greater than or equal to about 5% (w/v), greater than or equal to about 6% (w/v), greater than or equal to about 8% (w/v), greater than or equal to about 10% (w/v), greater than or equal to about 12% (w/v), greater than or equal to about 14% (w/v), greater than or equal to about 16% (w/v), greater than or equal to about 18% (w/v), or greater than or equal to about 20% (w/v)). The first concentration may be less than or equal to about 20% (w/v) (e.g., less than or equal to about 18% (w/v), less than or equal to about 16% (w/v), less than or equal to about 14% (w/v), less than or equal to about 12% (w/v), less than or equal to about 10% (w/v), less than or equal to about 8% (w/v), less than or equal to about 6% (w/v), less than or equal to about 5% (w/v), less than or equal to about 4% (w/v), less than or equal to about 3% (w/v), less than or equal to about 2% (w/v), or less than or equal to about 1.5% (w/v)). For example, the first concentration may be greater than or equal to about 1% (w/v) to less than or equal to about 20% (w/v) or optionally greater than or equal to about 1% (w/v) to less than or equal to about 5% (w/v).

In some aspects, the biomaterial also comprises a plurality of at least one type of living cells, such as undifferentiated or differentiated cells. The undifferentiated cells include progenitor cells, stem cells, embryonic stem cells, induced pluripotent stem cells, mesenchymal stem cells, epithelial stem cells, and combinations thereof, as non-limiting examples. The differentiated cells include epidermal cells, endothelial cells, fibroblasts, tissue specific cells, and organ or tissue specific cells, such as cells normally found in a breast, lung, liver, heart, brain, pancreas, prostate, cervix, ovary, bladder, gall bladder, kidney, colon, stomach, oral cavity, skin, tendon, ligament, cancer cells thereof, and combinations thereof, as non-limiting examples. In some aspects, the biomaterial also comprises a plurality of cell-derived products, such as extracellular vesicles.

In some aspects, the biomaterial also comprises an additive selected from the group consisting of at least one growth factor, at least one serum, at least one antimicrobial agent (e.g., antiseptic, antibiotic, antiviral, antifungal), at least one active pharmaceutical ingredient, at least one polynucleotide, at least one amino acid, and combinations thereof and/or an adjunct agent selected from the group consisting of preservatives, visualization markers, imaging agents, monitoring agents, agents for increasing efficiency, agents for increasing cytocompatibility, agents for increasing biocompatibility, agents for modulating an immune response, agents for providing an additional function to the biomaterial, agents for providing an additional function to the biomaterial, and combinations thereof.

The current technology also provides a method for making or preparing the biomaterial. The method comprises combining a first solution comprising GelMA, a linker (e.g., GPTMS), a photoinitiator, and a second solution comprising glass-ceramic precursors and Ag to form a biomaterial precursor solution and incubating the biomaterial precursor solution to form the biomaterial comprising the GelMA-AgBaG. After the incubating, the method can include adding at least one type of a plurality of living cells, a cellular product (e.g., ECM or ECM derivative), an additive, or an adjunct agent to the biomaterial, wherein the cells, additive, and adjunct agents are described above. The GelMA-AgBaG of the biomaterial is crosslinkable, for example, by illuminating with ultraviolet (UV) light (e.g., 100-400 nm) for greater than or equal to about 10 seconds to less than or equal to about 5 minutes. When crosslinked, the GelMA-AgBaG can define a construct having a 3D shape.

The incubating is performed at a first temperature for a first duration sufficient to form the biomaterial. The first temperature can be maintained in an oven, incubator, hotplate, or a water bath, as non-limiting examples. The first temperature may be greater than or equal to about 25° C. (e.g., greater than or equal to about 30° C., greater than or equal to about 35° C., greater than or equal to about 40° C., greater than or equal to about 45° C., greater than or equal to about 50° C., greater than or equal to about 55° C., greater than or equal to about 60° C., greater than or equal to about 65° C., greater than or equal to about 70° C., greater than or equal to about 75° C., greater than or equal to about 80° C., greater than or equal to about 85° C., greater than or equal to about 90° C., or greater than or equal to about 95° C.). The first temperature may be less than or equal to about 100° C. (e.g., less than or equal to about 95° C., less than or equal to about 90° C., less than or equal to about 85° C., less than or equal to about 80° C., less than or equal to about 75° C., less than or equal to about 70° C., less than or equal to about 65° C., less than or equal to about 60° C., less than or equal to about 55° C., less than or equal to about 50° C., less than or equal to about 45° C., less than or equal to about 40° C., less than or equal to about 35° C., or less than or equal to about 30° C.). For example, the first temperature may be greater than or equal to about 25° C. to less than or equal to about 100° C.

The first duration may be greater than or equal to about 0.5 hours (e.g., greater than or equal to about 1 hour, greater than or equal to about 2 hours, greater than or equal to about 3 hours, greater than or equal to about 4 hours, greater than or equal to about 5 hours, greater than or equal to about 6 hours, greater than or equal to about 7 hours, greater than or equal to about 8 hours, greater than or equal to about 9 hours, greater than or equal to about 10 hours, greater than or equal to about 11 hours, greater than or equal to about 12 hours, greater than or equal to about 13 hours, greater than or equal to about 14 hours, greater than or equal to about 15 hours, greater than or equal to about 16 hours, greater than or equal to about 17 hours, greater than or equal to about 18 hours, greater than or equal to about 19 hours, greater than or equal to about 20 hours, greater than or equal to about 21 hours, greater than or equal to about 22 hours, or greater than or equal to about 23 hours). The first duration may be less than or equal to about 24 hours (e.g., less than or equal to about 23 hours, less than or equal to about 22 hours, less than or equal to about 21 hours, less than or equal to about 20 hours, less than or equal to about 19 hours, less than or equal to about 18 hours, less than or equal to about 17 hours, less than or equal to about 16 hours, less than or equal to about 15 hours, less than or equal to about 14 hours, less than or equal to about 13 hours, less than or equal to about 12 hours, less than or equal to about 11 hours, less than or equal to about 10 hours, less than or equal to about 9 hours, less than or equal to about 8 hours, less than or equal to about 7 hours, less than or equal to about 6 hours, less than or equal to about 5 hours, less than or equal to about 4 hours, less than or equal to about 3 hours, less than or equal to about 2 hours, or less than or equal to about 1 hour). For example, the first duration may be greater than or equal to about 0.5 hours to less than or equal to about 24 hours or optionally greater than or equal to about 10 hours to less than or equal to about 20 hours.

The first solution comprises the GelMA dissolved in PBS at a second concentration of greater than or equal to about 50 mg/ml (5% (w/v)) (e.g., greater than or equal to about 75 mg/mL, greater than or equal to about 100 mg/mL, greater than or equal to about 120 mg/mL, greater than or equal to about 150 mg/mL, greater than or equal to about 175 mg/mL. The second concentration may be less than or equal to about 200 mg/ml (e.g., less than or equal to about 175 mg/mL, less than or equal to about 150 mg/mL, less than or equal to about 120 mg/mL, less than or equal to about 100 mg/mL, or less than or equal to about 75 mg/mL). For example, the second concentration may be greater than or equal to about 50 mg/ml (5% (w/v)) to less than or equal to about 200 mg/ml (20% (w/v)) or optionally greater than or equal to about 75 mg/mL (5% (w/v)) to less than or equal to about 150 mg/ml (20% (w/v)).

A first method (i.e., an aqueous method) of making the GelMA comprises incubating gelatin type A and MAA in a CB buffer having a pH of 7-9 or PBS having a pH of 7-9 to form a GelMA solution. The incubating is performed at a second temperature for a second duration sufficient to form the GelMA. The temperature can be maintained in an oven, incubator, hotplate, or a water bath, as non-limiting examples. The method then includes filtering the GelMA solution to remove impurities and dialyzing (e.g., 10-14 kDa cut off) the GelMA solution against water to remove toxic reagents. The method can then include freeze-drying, i.e., lyophilizing, the GelMA. To form the first solution, the GelMA is dissolved in phosphate buffered saline (PBS).

The second temperature may be greater than or equal to about 25° C. (e.g., greater than or equal to about 30° C., greater than or equal to about 35° C., greater than or equal to about 40° C., greater than or equal to about 45° C., greater than or equal to about 50° C., greater than or equal to about 55° C., greater than or equal to about 60° C., greater than or equal to about 65° C., greater than or equal to about 70° C., greater than or equal to about 75° C., greater than or equal to about 80° C., greater than or equal to about 85° C., greater than or equal to about 90° C., or greater than or equal to about 95° C.). The second temperature may be less than or equal about 100° C. (e.g., less than or equal to about 95° C., less than or equal to about 90° C., less than or equal to about 85° C., less than or equal to about 80° C., less than or equal to about 75° C., less than or equal to about 70° C., less than or equal to about 65° C., less than or equal to about 60° C., less than or equal to about 55° C., less than or equal to about 50° C., less than or equal to about 45° C., less than or equal to about 40° C., less than or equal to about 35° C., or less than or equal to about 30° C.). For example, the second temperature may be greater than or equal to about 25° C. to less than or equal to about 100° C.

The second duration may be greater than or equal to about 0.5 hours (e.g., greater than or equal to about 1 hour, greater than or equal to about 2 hours, greater than or equal to about 3 hours, greater than or equal to about 4 hours, greater than or equal to about 5 hours, greater than or equal to about 6 hours, greater than or equal to about 7 hours, greater than or equal to about 8 hours, greater than or equal to about 9 hours, greater than or equal to about 10 hours, or greater than or equal to about 11 hours). The second duration may be less than or equal to about 12 hours (e.g., less than or equal to about 11 hours, less than or equal to about 10 hours, less than or equal to about 9 hours, less than or equal to about 8 hours, less than or equal to about 7 hours, less than or equal to about 6 hours, less than or equal to about 5 hours, less than or equal to about 4 hours, less than or equal to about 3 hours, less than or equal to about 2 hours, or less than or equal to about 1 hour). For example, the second duration may be greater than or equal to about 0.5 hours to less than or equal to about 12 hours or optionally greater than or equal to about 1 hours to less than or equal to about 5 hours.

Because the first method uses dialysis to remove toxic reagents, which may cause the GelMA to undergo hydrolysis via random chain scission, thereby decreasing the molecular weight and performance of the GelMA in a unpredictable manner, the current technology also provides a second method (i.e., a non-aqueous method) of making the GelMA. The second method allows for dissolution of all reagents while preventing or minimizing the hydrolysis of the GelMA. Moreover, the second method does not require dialysis if a solvent is used having a low dielectric constant (i.e., ¿≤3 or ¿≤2) to increase attractive forces between oppositely-charged portions of the GelMA and cause aggregation through attractive electrostatic and dipole forces, thus precipitating out the GelMA; an ability to maintain dissolution of byproducts (e.g., DMSO and methacrylic anhydride); sufficient volatility at room temperature; a density lower than water; and minimal safety concerns. An exemplary solvent that fulfills the above criteria is toluene. The relationship between the dielectric constant and protein solubility can be quantified at the isoelectric point using Eq. 1:

$$log ( S ) = k / {e}^{2} + log ( {S}^{0} ) , \qquad Eq . 1$$

where S⁰ is an extrapolated value of S, e is the dielectric constant of the mixture, and k is a constant relating to the dielectric constant of water. The second method reduces processing time by up to about 95%, eliminates the dialysis step of the first method, and improves yield and reproducibility.

The second method comprises forming a gelatin solution by dissolving gelatin type A in DMSO at a third concentration and a third temperature. The third concentration may be greater than or equal to about 5% (w/v) (e.g., greater than or equal to about 6% (w/v), greater than or equal to about 7% (w/v), greater than or equal to about 8% (w/v), greater than or equal to about 9% (w/v), greater than or equal to about 10% (w/v), greater than or equal to about 11% (w/v), greater than or equal to about 12% (w/v), greater than or equal to about 13% (w/v), or greater than or equal to about 14% (w/v)). The third concentration may be less than or equal to about 15% (w/v) (e.g., less than or equal to about 14% (w/v), less than or equal to about 13% (w/v), less than or equal to about 12% (w/v), less than or equal to about 11% (w/v), less than or equal to about 10% (w/v), less than or equal to about 9% (w/v), less than or equal to about 8% (w/v), less than or equal to about 7% (w/v), or less than or equal to about 6% (w/v)). For example, the third concentration may be greater than or equal to about 5% (w/v) to less than or equal to about 15% (w/v).

The third temperature may be greater than or equal to about 25° C. (e.g., greater than or equal to about 30° C., greater than or equal to about 35° C., greater than or equal to about 40° C., greater than or equal to about 45° C., greater than or equal to about 50° C., greater than or equal to about 55° C., greater than or equal to about 60° C., greater than or equal to about 65° C., greater than or equal to about 70° C., greater than or equal to about 75° C., greater than or equal to about 80° C., greater than or equal to about 85° C., greater than or equal to about 90° C., or greater than or equal to about 95° C.). The third temperature may be less than or equal to about 100° C. (e.g., less than or equal to about 95° C., less than or equal to about 90° C., less than or equal to about 85° C., less than or equal to about 80° C., less than or equal to about 75° C., less than or equal to about 70° C., less than or equal to about 65° C., less than or equal to about 60° C., less than or equal to about 55° C., less than or equal to about 50° C., less than or equal to about 45° C., less than or equal to about 40° C., less than or equal to about 35° C., or less than or equal to about 30° C.). For example, the first temperature may be greater than or equal to about 25° C. to less than or equal to about 100° C.).

The second method then comprises adding MAA to the gelatin over a first time period to form the GelMA in the gelatin solution. The first time period may be greater than or equal to about 0.5 (e.g., greater than or equal to about 1 hour, greater than or equal to about 2 hours, greater than or equal to about 3 hours, greater than or equal to about 4 hours, or greater than or equal to about 5 hours). The first time period may be less than or equal to about 6 hours (e.g., less than or equal to about 5 hours, less than or equal to about 4 hours, less than or equal to about 3 hours, less than or equal to about 2 hours, or less than or equal to about 1 hour). For example, the first time period may be greater than or equal to about 0.5 hours to less than or equal to about 6 hours. The MAA can be added in increments of from about 1/10 to about ⅓, including increments of about 1/10, about ⅙, and about ⅓ over the time period.

The second method then comprises precipitating the GelMA by adding toluene to the gelatin solution at about 2 times to 5 times, e.g., about 2 times, about 3 times, about 4 times, or about 5 times, the volume of dimethyl sulfoxide (DMSO) in the gelatin solution. The second method next comprises decanting the DMSO to isolate the GelMA and washing the GelMA, for example, once, twice, or thrice, with water (with centrifuging prior to removing the water, for example, by aspirating or decanting). After the washing, the second method can include incubating the GelMA at about room temperature to allow at least a portion of any remaining toluene to evaporate, freezing GelMA in water at about −20° C. overnight, and lyophilizing the GelMA. To form the first solution, the GelMA is dissolved in PBS.

As discussed above, the photoinitiator can be VA-086, LAP, Irgacure 2959, and combinations thereof, as non-limiting examples. The photoinitiator is included in the biomaterial precursor solution at a fourth concentration of greater than or equal to about 1% (w/v) (e.g., greater than or equal to about 1.5% (w/v), greater than or equal to about 2% (w/v), greater than or equal to about 2.5% (w/v), greater than or equal to about 3% (w/v), greater than or equal to about 3.5% (w/v), greater than or equal to about 4% (w/v), greater than or equal to about 4.5% (w/v), greater than or equal to about 5% (w/v), greater than or equal to about 6% (w/v), greater than or equal to about 8% (w/v), greater than or equal to about 10% (w/v), greater than or equal to about 12% (w/V), greater than or equal to about 14% (w/v), greater than or equal to about 16% (w/v), or greater than or equal to about 18% (w/v)). The fourth concentration may be less than or equal to about 20% (w/v) (e.g., less than or equal to about 18% (w/v), less than or equal to about 16% (w/v), less than or equal to about 14% (w/v), less than or equal to about 12% (w/v), less than or equal to about 10% (w/v), less than or equal to about 8% (w/v), less than or equal to about 6% (w/v), less than or equal to about 5% (w/v), less than or equal to about 4.5% (w/v), less than or equal to about 4% (w/v), less than or equal to about 3.5% (w/v), less than or equal to about 3% (w/v), less than or equal to about 2.5% (w/v), less than or equal to about 2% (w/v), or less than or equal to about 1.5% (w/v)). For example, the fourth concentration may be greater than or equal to about 1% (w/v) to less than or equal to about 20% (w/v) or optionally greater than or equal to about 1% (w/v) to less than or equal to about 5% (w/v).

The second solution comprises the glass-ceramic precursors and Ag dissolved in an aqueous solvent, wherein the glass-ceramic precursors comprise a SiO₂ precursor (e.g., tetraethyl orthosilicate (TEOS)), a CaO precursor (e.g., calcium nitrate tetrahydrate (CaNT)), a P₂O₅ precursor (e.g., triethyl phosphate (TEP)), and optionally at least one of an Al₂O₃ precursor, a Na₂O precursor, or a K₂O precursor. In some aspects, the second solution is prepared by forming a BaG solution by combining greater than or equal to about 50 wt. % to less than or equal to about 70 wt. % SiO₂, greater than or equal to about 25 wt. % to less than or equal to about 40 wt. % CaO, and greater than or equal to about 5 wt. % to less than or equal to about 15 wt. % P₂O₅ in a solvent, such as water (from about 15 mL to about 30 mL in certain aspects), and a sol-gel porcelain solution stage by combining greater than or equal to about 50 wt. % to less than or equal to about 70 wt. % SiO₂, greater than or equal to about 1 wt. % to less than or equal to about 10 wt. % CaO, greater than or equal to about 1 wt. % to less than or equal to about 15 wt. % P₂O₅, and optionally at least one of greater than or equal to about 10 wt. % to less than or equal to about 20 wt. % Al₂O₃, greater than or equal to about 0 wt. % to less than or equal to about 15 wt. % Na₂O, greater than or equal to about 0 wt. % to less than or equal to about 15 wt. % K₂O, and greater than or equal to about 0 wt. % to less than or equal to about 10 wt. % Ag₂O in a solvent, such as water (from about 10 ml to about 20 mL in certain aspects). The BaG solution is then combined with the sol-gel porcelain solution to form the second solution comprising the glass-ceramic precursors. The second solution is included in the biomaterial precursor solution to result in total concentration of the glass-ceramic precursors of greater than or equal to about 0.1 mg/ml (e.g., greater than or equal to about 0.2 mg/mL, greater than or equal to about 0.3 mg/mL, greater than or equal to about 0.4 mg/mL, greater than or equal to about 0.5 mg/mL, greater than or equal to about 0.6 mg/mL, greater than or equal to about 0.7 mg/mL, greater than or equal to about 0.8 mg/mL, or greater than or equal to about 0.9 mg/mL). The total concentration may be less than or equal to about 1 mg/ml (e.g., less than or equal to about 0.9 mg/mL, less than or equal to about 0.8 mg/mL, less than or equal to about 0.7 mg/mL, less than or equal to about 0.6 mg/mL, less than or equal to about 0.5 mg/mL, less than or equal to about 0.4 mg/mL, less than or equal to about 0.3 mg/mL, or less than or equal to about 0.2 mg/mL). For example, the total concentration may be greater than or equal to about 0.1 mg/mL to less than or equal to about 1 mg/mL.

The current technology also provides a method of preparing a construct comprising crosslinked GelMA-AgBaG from the biomaterial. The construct can have a predetermined 3D geometry and can define, for example, a scaffold. The construct can be prepared on a benchtop or in a subject during a surgical procedure. The method comprises forming the biomaterial into the predetermined shape and crosslinking the GelMA-AgBaG, for example, by illuminating the biomaterial with UV light, as discussed above, to form the construct. In some aspects, the predetermined shape is formed by injecting or otherwise applying the biomaterial to a surface, such as a plastic, glass, or metal substrate, or a tissue (e.g., during a surgical procedure or injection) and then crosslinking the GelMA-AgBaG. In other aspects, the predetermined shape is formed by additive manufacturing, wherein the biomaterial is used as a bioink to 3D print a green structure by extrusion and then crosslink the GelMA-AgBaG in the green structure to form the construct having crosslinked GelMA-AgBaG. Therefore, the method can include generating a computerized model, for example, a computer-aided design (CAD) model, of a construct having a predetermined 3D shape or geometry that may be defined by a network or web of interconnected struts and having a predetermined porosity and average pore size defined by spaces between the struts. In a non-limiting example, the 3D shape or geometry comprises rows of substantially parallel struts, each row being stacked in a substantially orthogonal orientation onto a preceding row (with the exception of the first row).

In certain aspects, the porosity can be greater than or equal to about 40% (e.g., greater than or equal to about 45%, greater than or equal to about 50%, greater than or equal to about 55%, greater than or equal to about 60%, greater than or equal to about 65%, greater than or equal to about 70%, greater than or equal to about 75%, greater than or equal to about 80%, or greater than or equal to about 85%). The porosity may be less than or equal to about 90% (e.g., less than or equal to about 85%, less than or equal to about 80%, less than or equal to about 75%, less than or equal to about 70%, less than or equal to about 65%, less than or equal to about 50%, less than or equal to about 55%, less than or equal to about 50%, or less than or equal to about 45%). For example, the porosity may be greater than or equal to about 40% to less than or equal to about 90%.

The average pore size can be greater than or equal to about 200 μm (e.g., greater than or equal to about 250 μm, greater than or equal to about 300 μm, greater than or equal to about 350 μm, greater than or equal to about 400 μm, greater than or equal to about 450 μm, greater than or equal to about 500 μm, greater than or equal to about 550 μm, greater than or equal to about 600 μm, greater than or equal to about 650 μm, greater than or equal to about 700 μm, greater than or equal to about 750 μm). The average pore size may be less than or equal to about 800 μm (e.g., less than or equal to about 750 μm, less than or equal to about 700 μm, less than or equal to about 650 μm, less than or equal to about 600 μm, less than or equal to about 550 μm, less than or equal to about 500 μm, less than or equal to about 450 μm, less than or equal to about 400 μm, less than or equal to about 350 μm, less than or equal to about 300 μm, or less than or equal to about 250 μm). For example, the average pore size may be greater than or equal to about 200 μm to less than or equal to about 800 μm or optionally greater than or equal to about 200 μm to less than or equal to about 400 μm,

The struts can have a strut thickness or diameter of greater than or equal to about 50 μm (e.g., greater than or equal to about 100 μm, greater than or equal to about 150 μm, greater than or equal to about 200 μm, greater than or equal to about 250 μm, greater than or equal to about 300 μm, greater than or equal to about 350 μm, greater than or equal to about 400 μm, or greater than or equal to about 450 μm), The struct thickness or diameter may be less than or equal to about 500 μm (e.g., less than or equal to about 450 μm, less than or equal to about 400 μm, less than or equal to about 350 μm, less than or equal to about 300 μm, less than or equal to about 250 μm, less than or equal to about 200 μm, or less than or equal to about 150 μm). For example, the strut thickness or diameter may be greater than or equal to about 50 μm to less than or equal to about 500 μm or optionally greater than or equal to about 100 μm to less than or equal to about 300 μm,

The current technology also provides a method of treating a disorder or augmenting a structure or function of a tissue in a subject in need thereof using the construct described above. The subject can be a human or nonhuman mammal, bird, fish, reptile, or amphibian. The method comprises disposing the construct on a location or site of the disorder or the tissue in the subject. The construct can be fabricated prior to the disposing (as described above) or in situ. For example, the biomaterial can be injected or applied to the location or site of the disorder and then illuminated by UV light at the location or site to crosslink the GelMA-AgBaG of the biomaterial and form the construct.

In some aspects, the disorder is a bone defect, and the method comprises disposing the construct in a site of the bone defect. The construct can be additively manufactured from a CAD model generated from images (e.g., magnetic resonance imaging (MRI), computed tomography (CT), positron emission tomography (PET), ultrasound, X-ray, or combinations thereof) to be patient-specific, such that the construct has a mating surface that is a negative of the bone defect and nestingly mates to or rests against a corresponding surface of the defect in the bone in only a single orientation.

In other aspects, the disorder is a hemorrhage or aneurysm, and the method comprises disposing the construct at a source of the hemorrhage or at the aneurysm.

In other aspects, the disorder is a skin defect, and the method comprises disposing the construct on the skin defect. The skin defect can be, for example, a wound (e.g., acute or chronic), a burn, a pressure sore, or an excision. Because the skin defect may be at risk of being infected, the construct can prevent, minimize, or inhibit the formation of an infection at the wound or burn.

In other aspects, the disorder is inflamed tissue associated with osteomyelitis, periimplantitis, periodontitis, or combinations thereof, and the method comprises disposing the construct at the site of the inflamed tissue.

The current technology also provides a method of treating a subject having a risk of developing an infection. The method comprises disposing the construct at a site of the subject at risk of developing the infection. The construct prevents, minimizes, or inhibits the formation of the infection at the site.

Embodiments of the present technology are further illustrated through the following non-limiting examples.

### Example 1

GelMA is useful for mimicking an ECM, is suitable for loading many different organic and inorganic components, and can be used in combination with BaG for, for example, bone tissue regeneration. As shown in Table 1, the rheological properties of GelMA makes it suitable for bioinks that can be used in additive manufacturing processes. Developing GelMA-AgBaG hybrid hydrogel biomaterials for cell-laden bioprinting of 3D constructs, for example, scaffolds, can facilitate four-dimensional bioprinting.

TABLE 1
Experimental and literature values for the
rheological properties of GelMA bioinks.

| | GelMA-AgBaG | | | GelMA Bioink | | | |
|---|---|---|---|---|---|---|---|
| | Bioink (10% w/v | | | (10% w/v) | | | Recommended |
| Property | GelMA) | | | Literature | | | Literature |
| Viscosity (Pa · s) | 40,000 | ~1,000 | ~50 | ~300 | ~170 | ~40 | 0.03-60,000 |
| Shear Rate (s⁻¹) | 0.1 | 1 | 10 | 0.1 | 1 | 10 | — |

| Storage Modulus | ~75,000 | ~1,000 | 100-1,000 |
|---|---|---|---|
| (Pa) | | ~800 | |
| Loss Modulus | ~20,000 | ~5 | — |
| (Pa) | | ~40 | |

| | | | |
|---|---|---|---|
| Shear Thinning | ✓✓✓ | ✓✓✓ | ✓✓✓ |

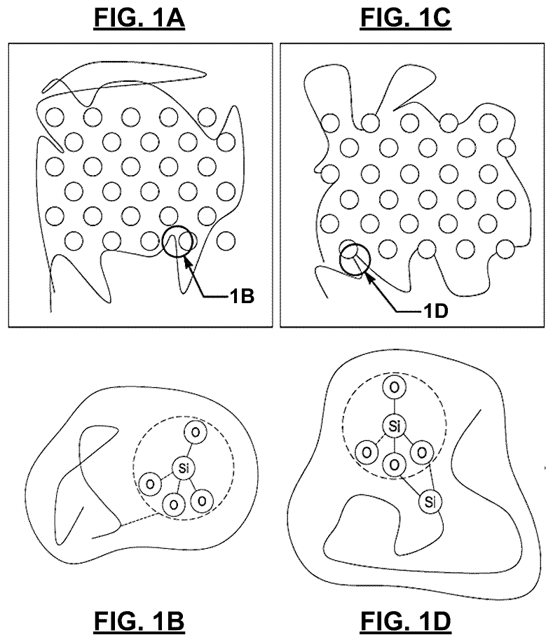

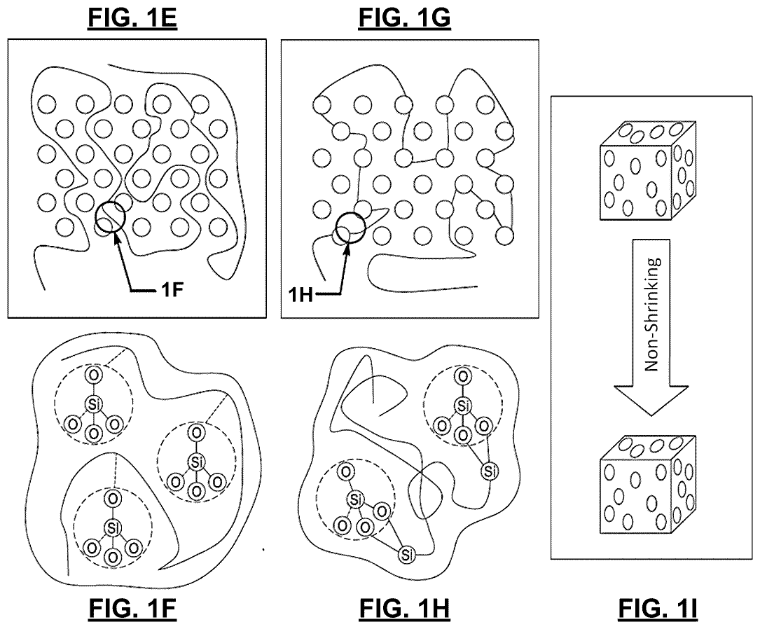

FIGS. 1A-1F show classes of hydrogel hybrid materials in accordance with various aspects of the present disclosure.

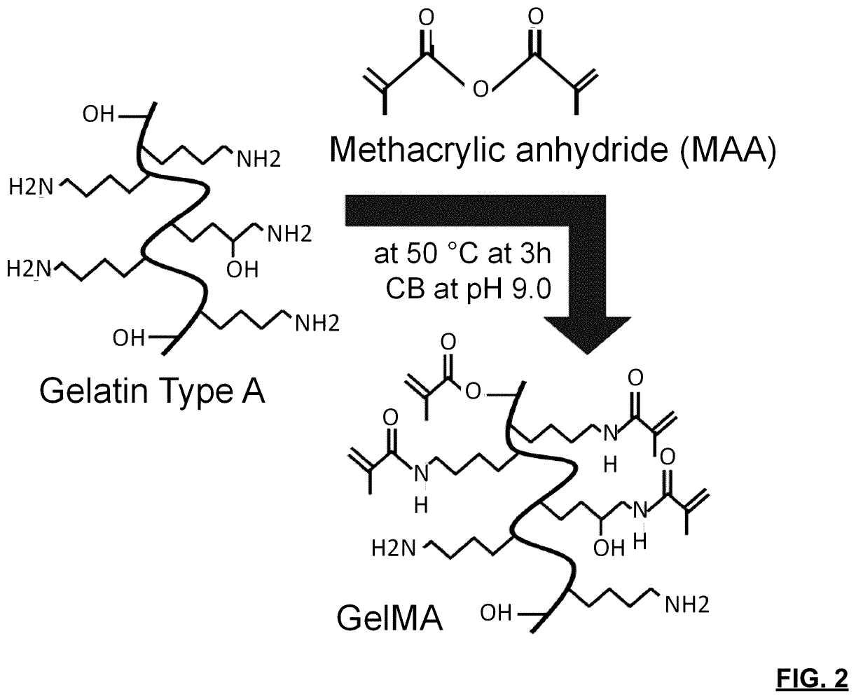

The availability of free amino groups increases in gelatin type A as pH increases from pH 7 to pH 9. Therefore, GelMA is made using a CB buffer system, pH 9. As shown in FIG. 2, MAA is added to a solution comprising gelatin type A and CB buffer system to form a reaction mixture, and the reaction mixture is incubated at about 50° C. for about 3 hours to form the GelMA.

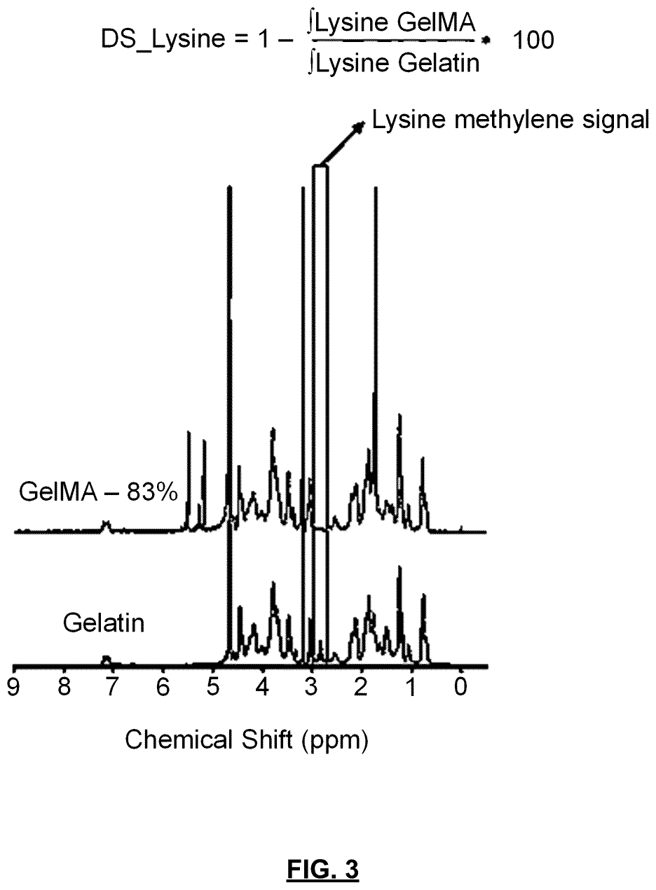

As shown in FIG. 3, ¹H-NMR is used to determine a degree of substitution (DS). The spectra of FIG. 3 are normalized to a phenylalanine signal (7.5-6.9 ppm), and an integral of lysine methylene signal (2.95-2.8 ppm) is used to calculate DS. The GelMA DS is determined to be 83%.

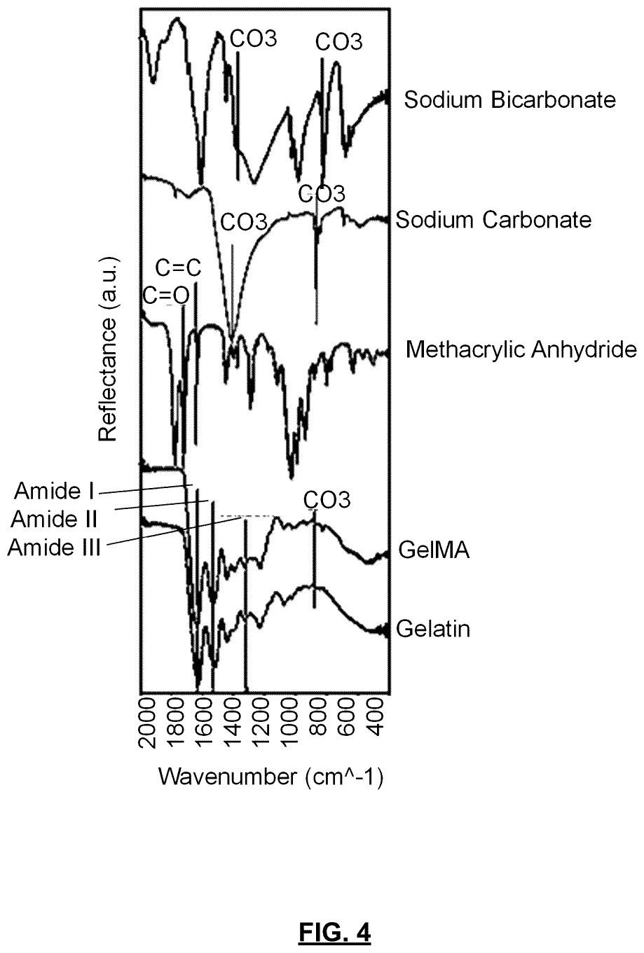

As shown in FIG. 4, FTIR of the GelMA shows minimal reagents, which suggests that the GelMA is adequately purified.

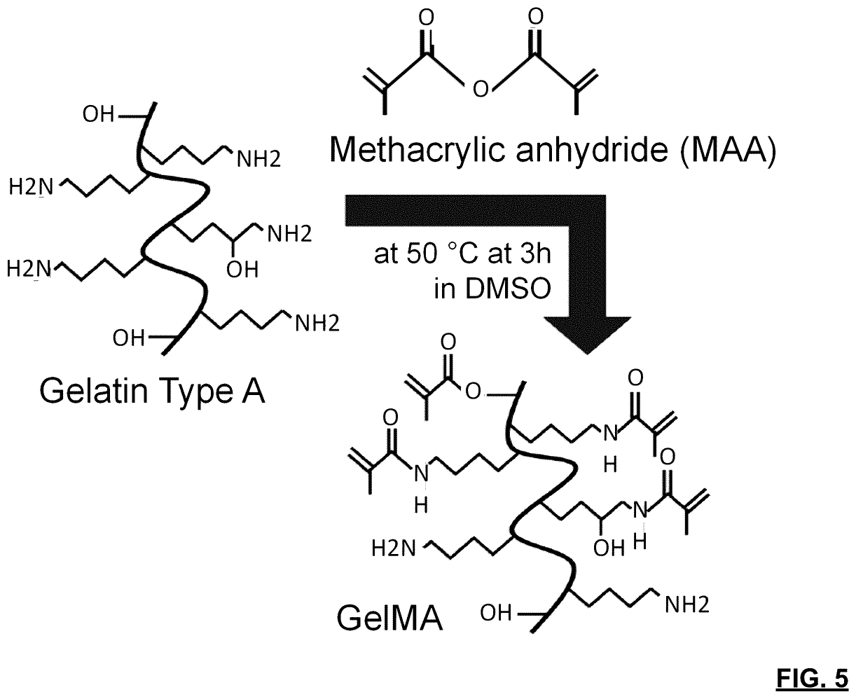

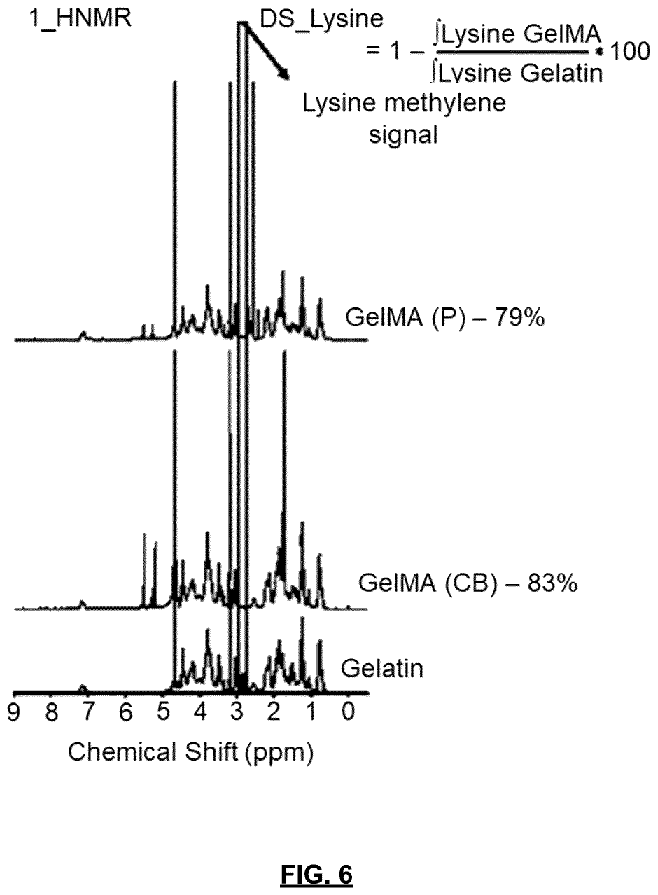

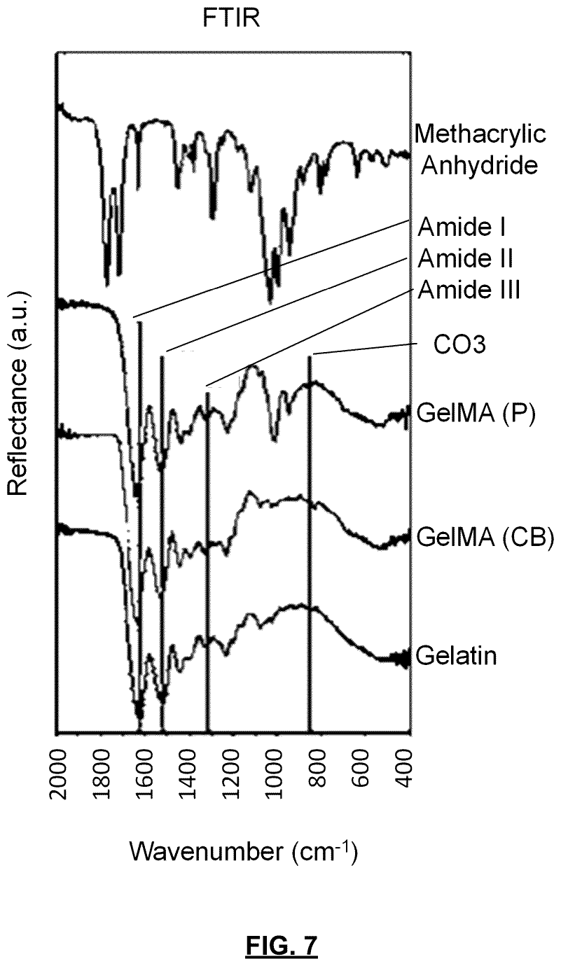

As shown in FIG. 5, GelMA is also made by dissolving 10% (w/v) gelatin type A in DMSO at 50° C. MAA is added over the course of 3 hours in ⅙ increments of the total volume to form GelMA in the DMSO. Toluene is then added to the GelMA-DMSO at 3 times the volume of the DMSO used to precipitate the GelMA. The DMSO and toluene are then decanted and the GelMA is washed thrice with distilled water (with centrifuging before aspirating the distilled water). After the third wash, the GelMA is incubated at room temperature to allow residual toluene to evaporate. The GelMA is then frozen as an aqueous solution at 20° C. overnight and then lyophilized. FIG. 6 shows ¹H-NMR spectra of the GelMA, and FIG. 7 shows FTIR of the GelMA.

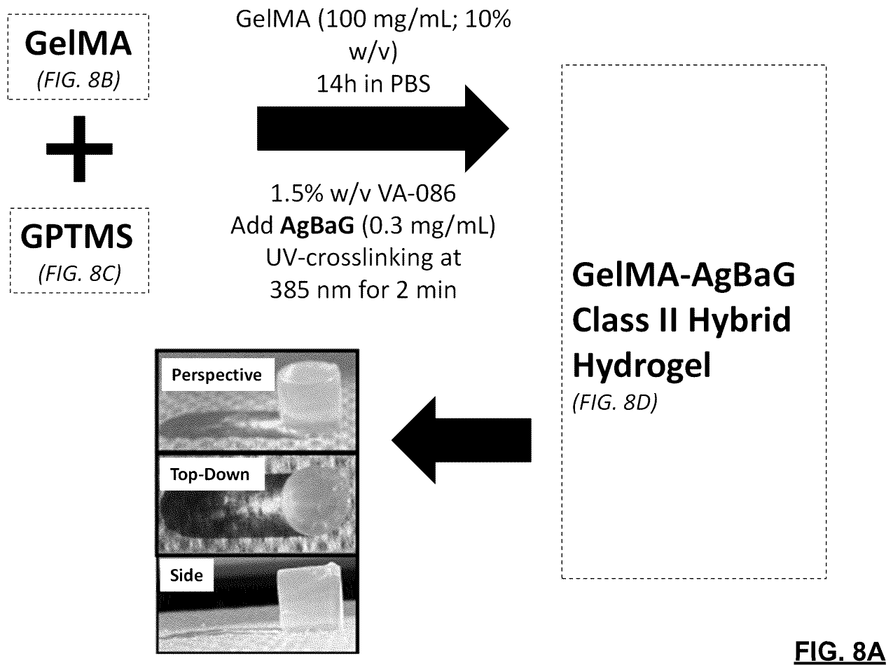

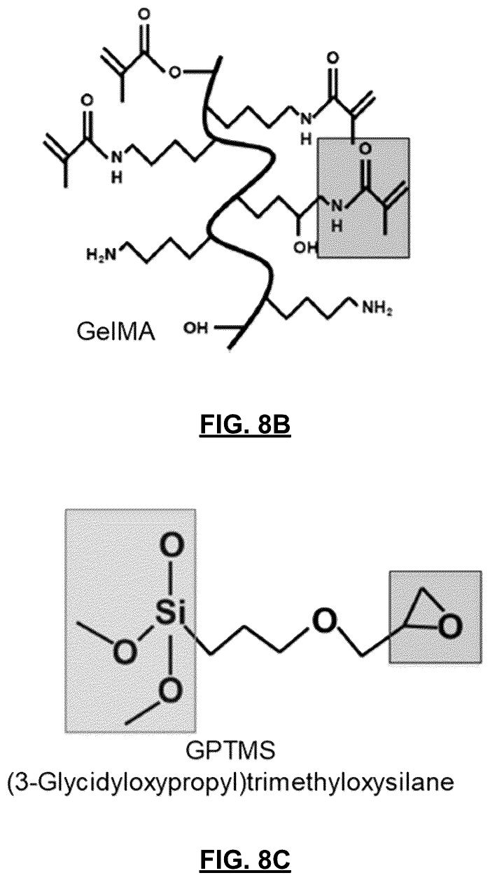

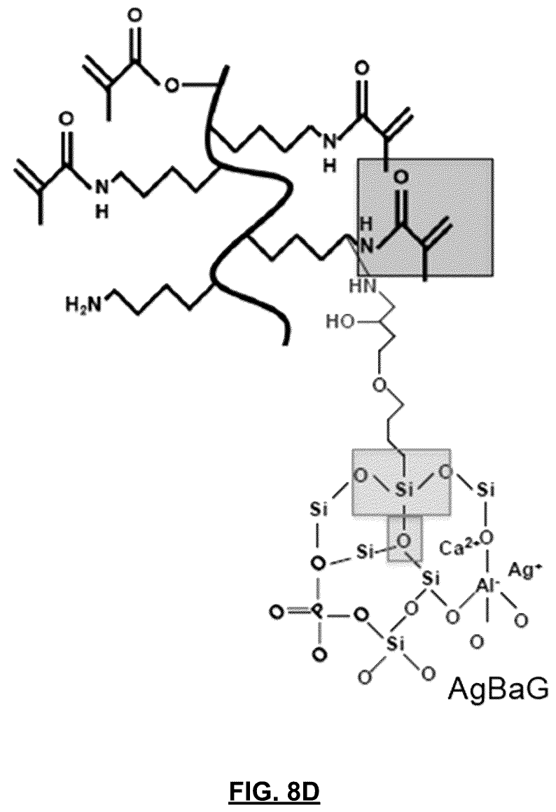

As shown in FIGS. 8A-8D, GelMA-AgBaG class II hybrid hydrogel (FIG. 8D) is generated by forming a GelMA solution comprising 100 mg/mL GelMA (FIG. 8B) in PBS and adding GPTMS (FIG. 8C) to the GelMA solution, along with 1.5% (w/v) VA-086 and 0.3 mg/mL Ag-BaG to form a reaction mixture. The reaction mixture is incubated at 50° C. for about 14 hours. The reaction mixture is then illuminated with UV light (385 nm) for about 2 minutes to form the GelMA-AgBaG.

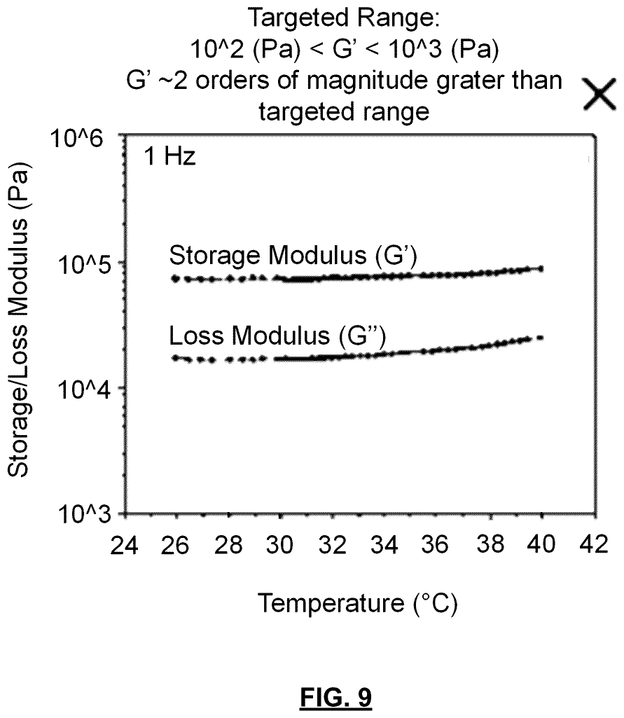

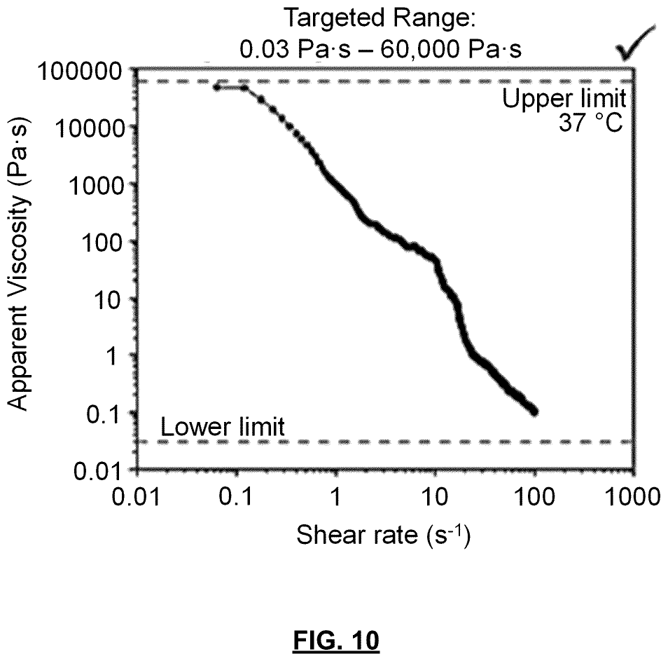

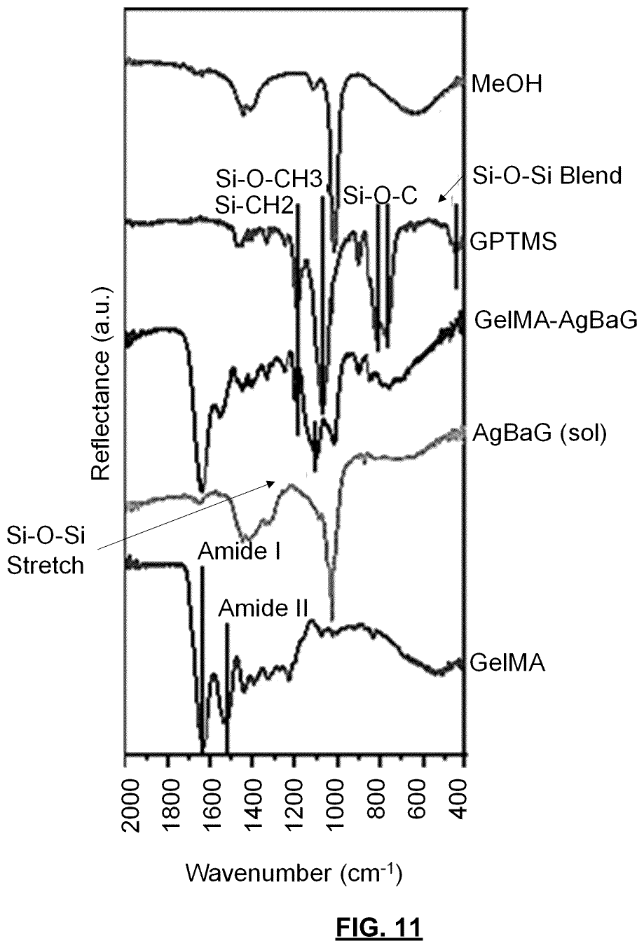

FIG. 9 is a graph showing that the storage modulus of the GelMA is two orders of magnitude greater than values provided in literature. FIG. 10 is a graph showing that the viscosity of the GelMA-AgBaG class II hybrid hydrogel is within an acceptable range, given that the applied shear rate is less than 100 s⁻¹. A decrease in viscosity as a function of shear rate indicates shear thinning behavior for the GelMA-AgBaG Class II hybrid hydrogel. Shear thinning behavior indicates that the hydrogel will exhibit self-healing abilities after removal of shear forces. Power fitting within the range of approximately 1-10 s⁻¹ is used to determine flow consistency index (K) and flow behavior index (n) for Eq. 2:

$${η}_{app} = K {γ}^{( n - 1 )} , \qquad Eq . 2$$

where K=417±10 (Pa·sⁿ), n=0.045±0.007, and R²=0.98. The flow behavior index (n) indicates that the GelMA-AgBaG hybrid hydrogel is highly shear thinning (i.e., non-Newtonian fluid, pseudoplastic).

As shown in FIG. 11, both GelMA and Ag-BaG structures are observed in FTIR of the GelMA-AgBaG. When the detection of Si—O—Si bonds is weak, Ag-BaG incorporation may be low due to minimal interaction time. A lack of Si—O—Si rocking at about 450 cm⁻¹ indicates an absence of cyclic Si—O—Si members, which may indicate that the glass network exists as linear chains.

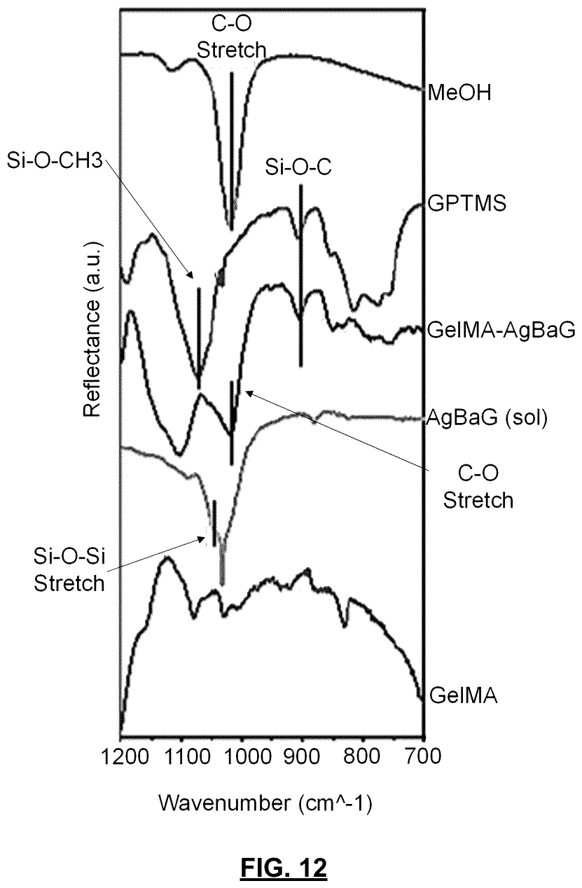

FIG. 12 provides additional FTIR spectra showing that methanol is present in the GelMA-AgBaG hybrid hydrogel and the C—O stretch peak aligns well. For AgBaG (sol), a sharp peak is identified at about 1025 cm⁻¹ and Si—O—Si stretching is identified at about 1050 cm⁻¹ (shoulder). No Si—O—CH₃ peak is noted in the hydrogel, which also indicates an absence of methyl groups and is indicative of successfully coupling AgBaG to GelMA via GPTMS.

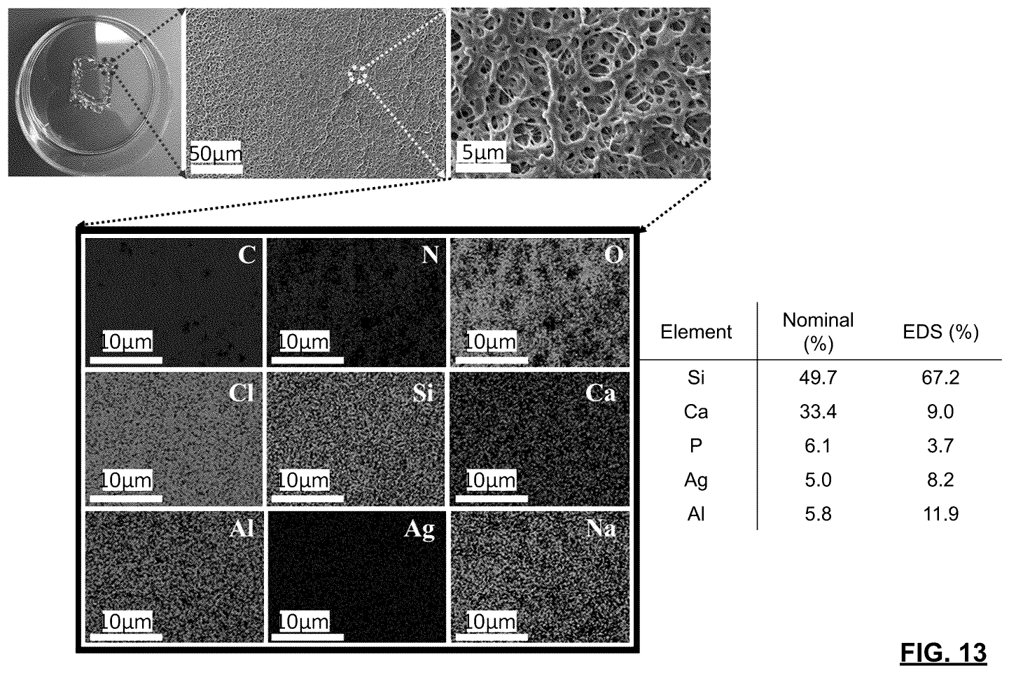

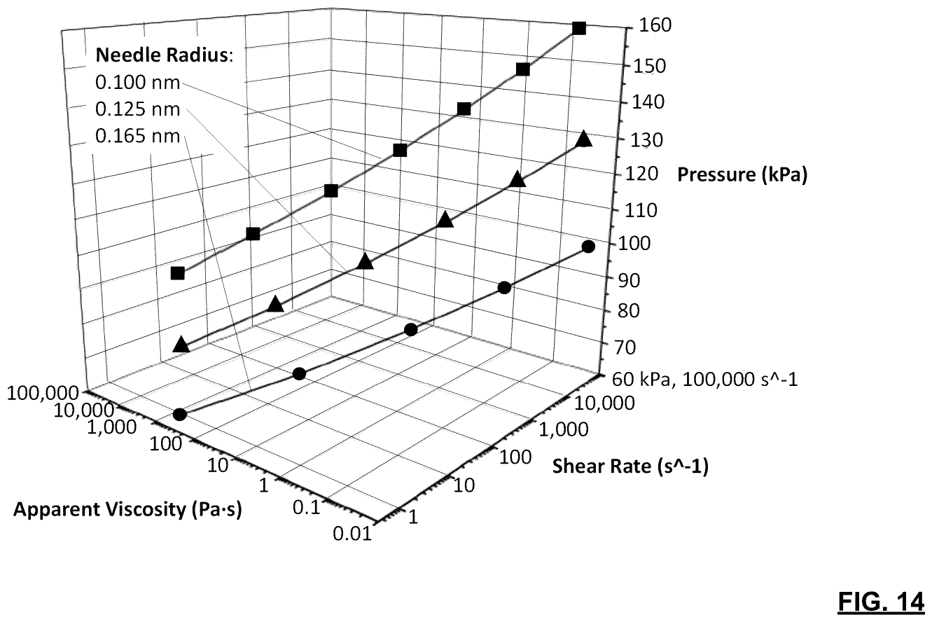

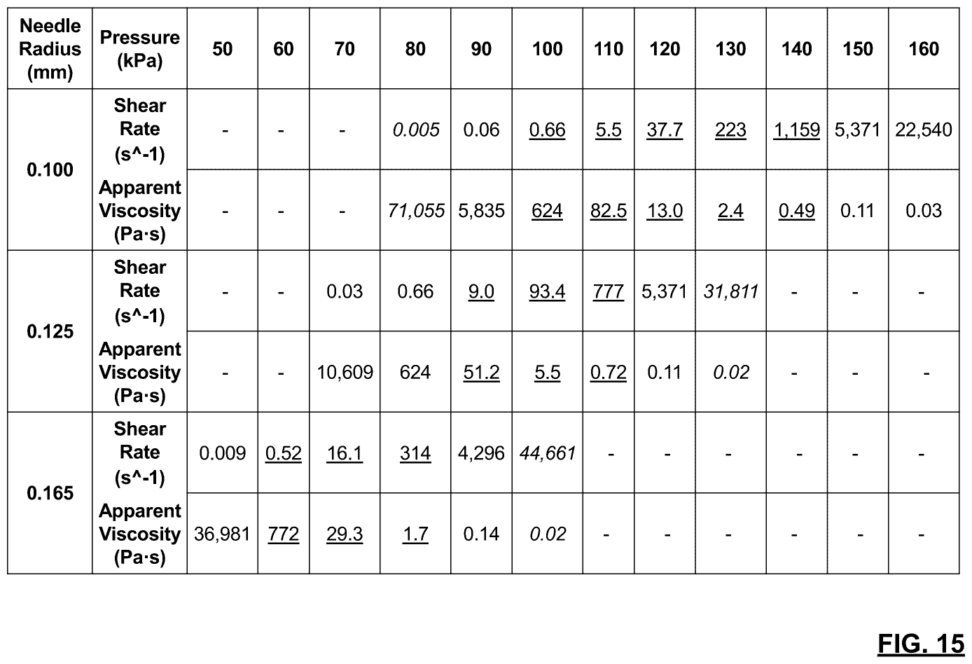

FIG. 13 shows SEM micrographs and EDS images of the GelMA-AgBaG. The micrographs and images show that the process of critical point drying is successful at preparing GelMA-AgBaG. Further, elemental homogeneity is observed in all regions at all magnifications, down to the micron level. Therefore, the fabrication method successfully produces GelMA-AgBaG with an elementally homogenous microstructure.

FIG. 14 is a plot showing the predicted shear rate and apparent viscosity based on various pressures and needle geometry. For all three needle geometries, reasonable windows of printability could be identified (see FIG. 15). The smaller the needle radius, the greater the window of printability.

FIG. 15 shows theoretical shear rate and viscosity for GelMA-AgBaG hybrid hydrogels calculated using the shear thinning coefficients (i.e., n and K), hypothetical pressures, and hypothetical needle geometry. Black represents values unreasonable to calculate, italics indicate values falling outside the printability range for apparent viscosity (i.e., 0.03-60,000 Pa·s), underlining indicates potential values for use, and plain text represents values that are likely sub-optimal.

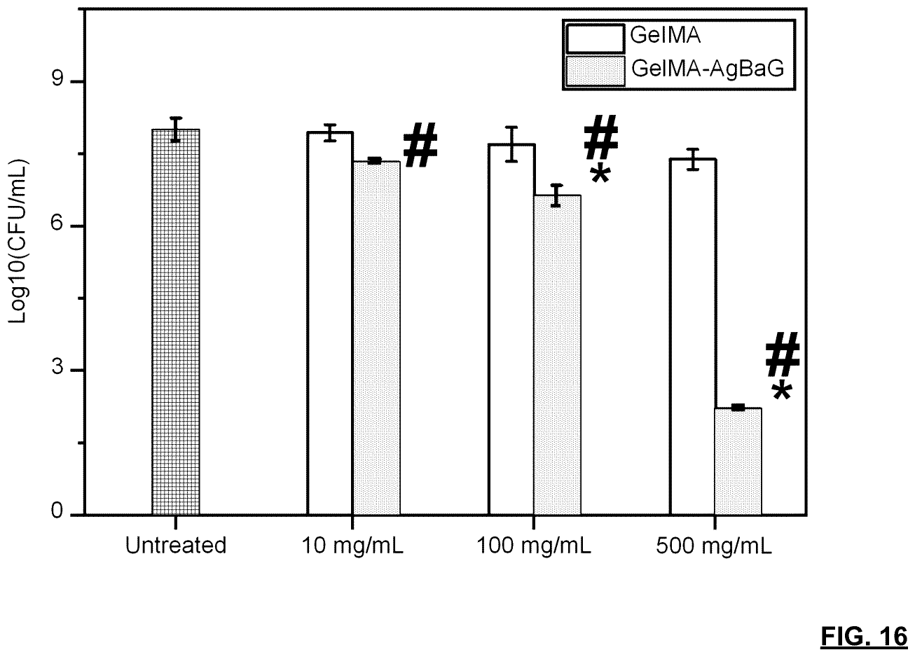

FIG. 16 shows that GelMA-AgBaG has antibacterial activity at concentrations between 10 mg/mL and 500 mg/mL.

### Example 2

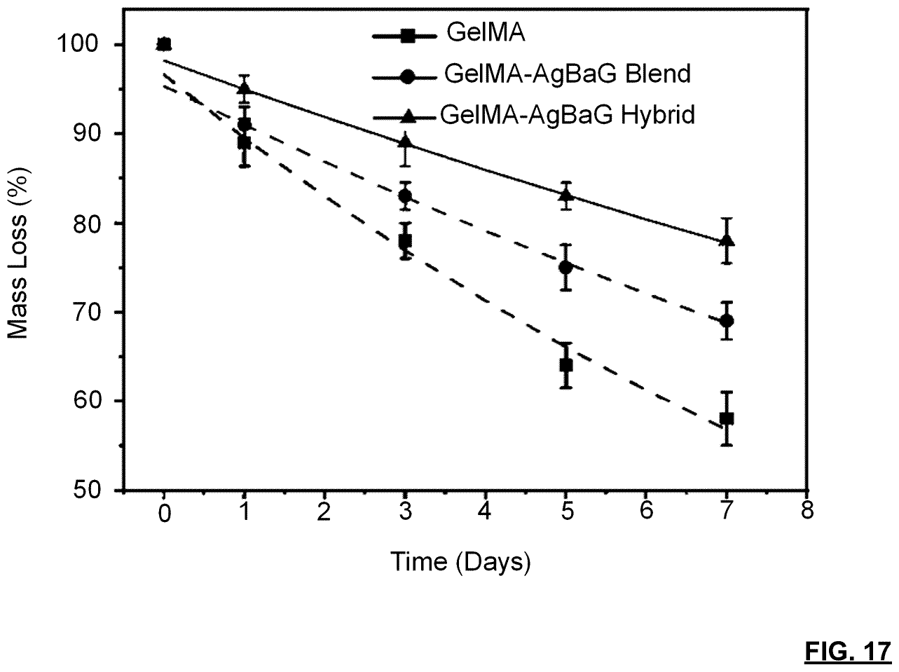

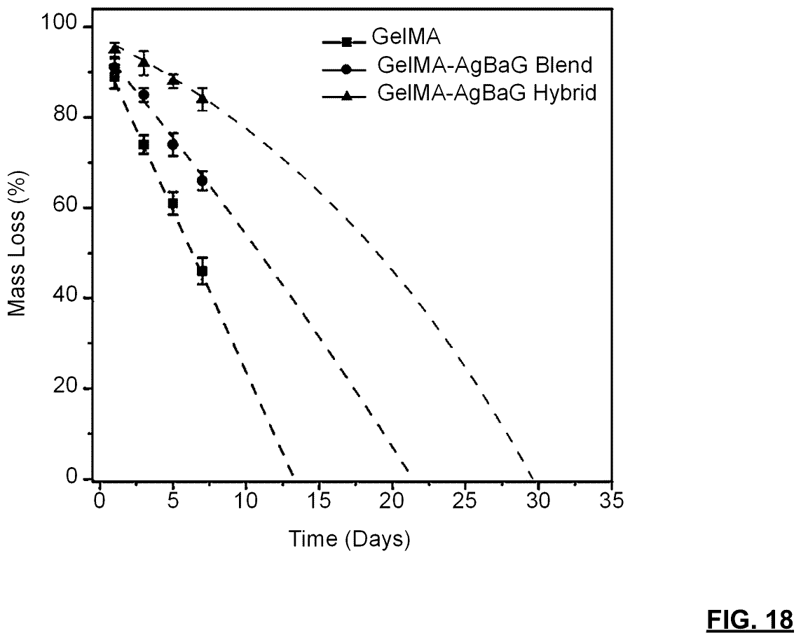

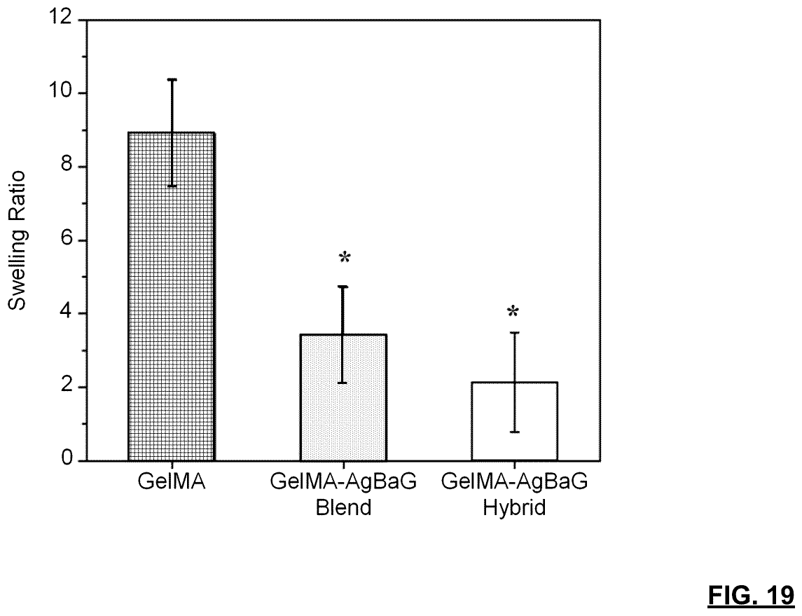

In another example, three sample groups are evaluated: GelMA, GelMA-AgBaG blend (where the AgBaG is physically mixed with GelMA), and GelMA-AgBaG hybrid (according to the current technology). All samples are immersed in distilled water to remove any impurities. The samples are then frozen and lyophilized. To determine swelling ratios, dry samples are immersed in distilled water at a concentration of 10% (w/v). For mass loss determinations, the dry mass of all the samples is recorded, and the samples are immersed in 0.05 M tris(hydroxymethyl)aminomethane (TRIS) buffer at a mass:volume ratio of 3.33. Samples are immersed for 1, 3, 5, and 7 days at 37° C. under 175 RPM shaking. The samples are extracted from solution, frozen, and lyophilized to determine the dry mass of the samples after immersion in the TRIS buffer. As shown in Table 2 and FIGS. 17-18, over the duration of the mass loss example, the GelMA loses the most mass, while the GelMA-AgBaG hybrid loses lose the least amount of mass. Evidence supports that the method of incorporation of the AgBaG (blend versus hybrid) affects the mass loss of the resulting material. As shown in FIG. 19, the swelling ratio is the smallest for the GelMA-AgBaG (hybrid) and greatest for GelMA alone.

TABLE 2
Mass loss fitted using exponential decay (i.e., y = ab^{x}, R² > 0.98).

| | | Estimated time to |
|---|---|---|
| | Rate of Mass Loss | complete degradation |
| | (% day⁻¹) | (days) |
| GelMA | 7.6 | 13 |
| GelMA-Ag-BaG Blend | 4.7 | 21 |
| GelMA-Ag-BaG Hybrid | 3.3 | 30 |

### Example 3

This example describes a bioink for improved 3D bioprinting of bone-like constructs.

### Introduction

Bone tissue loss can occur due to disease, trauma or following surgery, in each case treatment involving the use of bone grafts or biomaterials may be beneficial. Recent development of 3D bioprinting (3DBP) has facilitated the printing of customized bone substitutes. Bioinks used for bone 3DBP employ various particulate phases such as ceramic and bioactive glass particles embedded in the bioink creating a composite. When composite bioinks are used for 3DBP based on extrusion, particles are heterogeneously distributed causing damage to cells due to stresses created during flow in the matrix of the composite. Therefore, the objective of this example is to develop cell-friendly osteopromotive bioink mitigating the risk of cell damage due to the flow of particles. Towards this end, linked organic and inorganic components according to various aspects of the present disclosure, GelMA and Ag-doped bioactive glass (Ag-BaG), produce a hybrid material, GelMA-Ag-BaG (GAB). The distribution of the elements present in the Ag-BaG in the resulting hybrid GAB structure is examined. Rheological properties of the resulting hydrogel and its printability, as well as the degree of swelling and degradation over time, are also evaluated. GAB is compared to GelMA alone and GelMA-Ag-BaG nanocomposites. Results show the superiority of the hybrid GAB bioink in terms of homogenous distribution of the elements in the structure, rheological properties, printability, and degradation profiles. Accordingly, this new bioink represents a major advance for bone 3DBP.

A major clinical challenge in orthopedic and craniomaxillofacial (CMF) surgery is the development of critically sized bone defects typically caused by congenital malformations, trauma, infection, cancer, or surgical resection. Multiple treatment approaches have been implemented to address such defects such as the use of allographs, however, an important approach continues to be the use of autografts. Globally, over two million bone graft procedures are performed annually with the commonality of such procedures being second only to blood transfusions. The use of autografts is, however, limited by availability, donor-site morbidity, and the challenges of creating the required shape (e.g. CMF applications). While the use of allografts can address the resource limitations and eliminate concerns regarding donorsite morbidity associated with the use of autografts, the use of allografts presents its unique challenges. For example, the use of devitalized allographs not only employs a high-cost laborious process, but also results in limited revascularization significantly increasing the risk of incurring an immunological reaction and infection. Treatment strategies for addressing infected bone often require a second revision surgery that not only prolongs recovery time but also has the potential to lead to more severe ramifications such as permanent loss of function and even amputation. Therefore, it would be beneficial to develop effective alternative approaches.

To address this, biomaterials have been used, classically as acellular constructs with a focus on identifying biocompatible and bioinert materials. Success has been demonstrated in addressing the shortcomings of traditional biomaterials-based strategies by tackling said challenges through more biomimetic approaches. The advantages of using cell-seeded polymeric tissue engineering constructs are demonstrated in various studies. Unfortunately, many of the available polymers are not cell-friendly due to their synthetic origins. Improvements to the cell-friendly nature of biomaterials have been realized through the use of naturally-derived biomaterials such as decellularized tissue matrices or natural polymers such as collagen and gelatin) either incorporated in conjunction with other biomaterials such as synthetic polymers and growth factors or used alone. The enhanced cell-friendly characteristics are in part due to the presence of the RGD peptide sequences in the naturally derived biomaterials known to be an important factor in cell attachment and function. While acellular biomaterial-based approaches have represented important advances in the biomaterials field, the inclusion of cells appears to be important to further advancements. Furthermore, the use of cell seeding alone has proven to be give rise to challenges given the subsequent premature failure of implants due to inhomogeneous cell distributions.

3D printing (3DP) technologies allow significant advancements to be made in controlling the geometry of tissue engineering. Combined with imaging and design technologies such as CT, MRI, and CAD, 3DP facilitated the development of customized bone substitutes. An important innovation in the expansion of 3DP technologies for tissue engineering was the advent of 3DBP allowing cell-laden constructs to be 3D printed leading to advances in the engineering of biomimetic living constructs. 3DBP patient-specific constructs show potential for improving treatment outcomes and accelerating the time of recovery.

The creation of a printable bioink can be achieved through the incorporation of cells into a liquid matrix that undergoes solidification post-print, forming the desired tissue-like constructs. Most commonly, 3DBP uses extrusion-based methods for the printing process; however such methods are known to reduce cell viability due to the introduction of shear forces during extrusion. The incorporation of solid elements into bioinks such as glass or ceramic particles that may support bone growth, osteopromotive, leads to increased shear forces during 3DBP leading to additional decreases in cell viability.

Typically, bioinks used for 3DBP of bone-like constructs incorporate osteopromotive elements such as Ca-based bioceramics or silicate-based biomaterials in the form of either micro-sized or nano-sized particles. The particles are introduced into the cell-laden polymer matrix through mixing allowing composites to be 3DBP. For example, nanoparticles (NPs) of silicate glasses have been combined with GelMA to form nanocomposites with the two components held together by ionic interactions. Such nanocomposite bioinks are limited to the degree of homogeneity they can achieve not only by the size of the particles incorporated but also by the degree of agglomeration. This is evidenced by the increase in dead osteoblasts after the extrusion 3DBP of BaG containing cell-laden bioinks as a result of the increased shear forces introduced by the BaG particles. Therefore, alternative strategies to incorporating osteopromotive elements would be beneficial.

An innovative approach to overcoming these limitations is to chemically link the osteopromotive component(s) to the polymer matrix. It is expected that the chemical bonding of the osteopromotive component(s) and polymer matrix will result in a stable structure allowing agglomeration of osteopromotive component(s) to be minimized. Furthermore, to achieve the greatest degree of homogenization between the osteopromotive component(s) and the polymer matrix will likely utilize an in situ synthesis method. Combining these factors should deliver 3DBP bone-like constructs, where the body cannot distinguish the individual components used in the bioink. This would allow for advanced material characteristics to be achieved that could not otherwise have been realized using a composites approach.

BaG is an attractive osteopromotive component for incorporation into cell-laden bioinks for 3DBP of bone-like constructs given its well-documented improvements in cell viability, osteogenic differentiation, antibacterial properties, and in vivo cell survival, as well as anti-inflammatory, and pro-angiogenic properties. The ideal BaG-containing bioink, therefore, is likely to exhibit the following characteristics: (1) supports the optimal or improved osteogenic response for cells to be induced as a result of the ions provided by the BaG, (2) increases the stiffness of the 3DBP bone-like constructs, possesses the sufficient anti-inflammatory characteristics for healing, and (4) allows for the promotion of angiogenesis.

We, therefore, aim to utilize an innovative approach that combines GelMA with an Ag-doped GAB to deliver a novel osteopromotive and antibacterial material. To achieve this, we used GelMA as the polymer matrix to both facilitate crosslinking and enhance cell attachment and function. Ag-BaG is selected for its osteogenic, angiogenic, and antibacterial properties, along with the ability of the Ag-BaG to enhance the strength of the delivered material. The GelMA and Ag-BaG are then chemically linked to produce the hybrid, GAB. Additionally, Ag-BaG NPs are mixed with GelMA to deliver a nanocomposite material, where the developed GAB hybrid material is found to be superior to the synthesized nanocomposite material demonstrating the effectiveness of this approach.

### Materials and Methods

### Material Synthesis

To synthesize GelMA, type A gelatin (300 bloom; Millipore Sigma) is dissolved in dimethyl sulfoxide (DMSO; Millipore sigma) at 50° C. and stirred at 500 RPM. Methacrylic anhydride (MAA; Millipore Sigma) is added in ⅙th increments every 30 minutes to achieve a final MAA:lysine ratio of 2.2. The resulting solution is then added to toluene at 3× the reaction volume used to induce precipitation of the GelMA. The solution is then decanted after 24h, washed thrice with distilled water, and then dissolved in distilled water at 50° C. After dissolution, the solution is placed at 37° C. for 24h to keep the GelMA dissolved while allowing for any remaining toluene to evaporate. The solution is then frozen and lyophilized before storage.

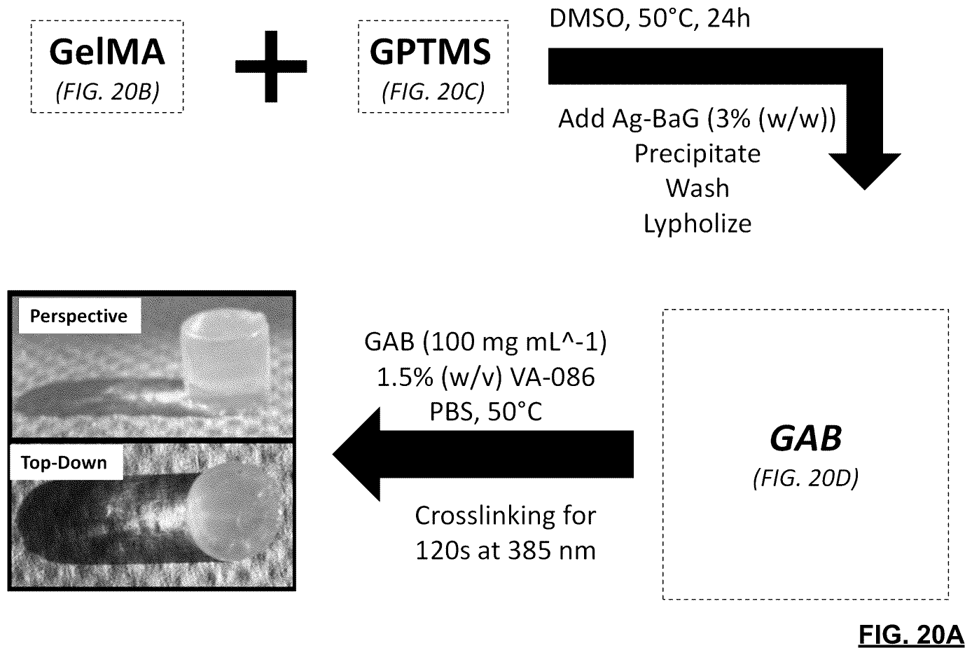

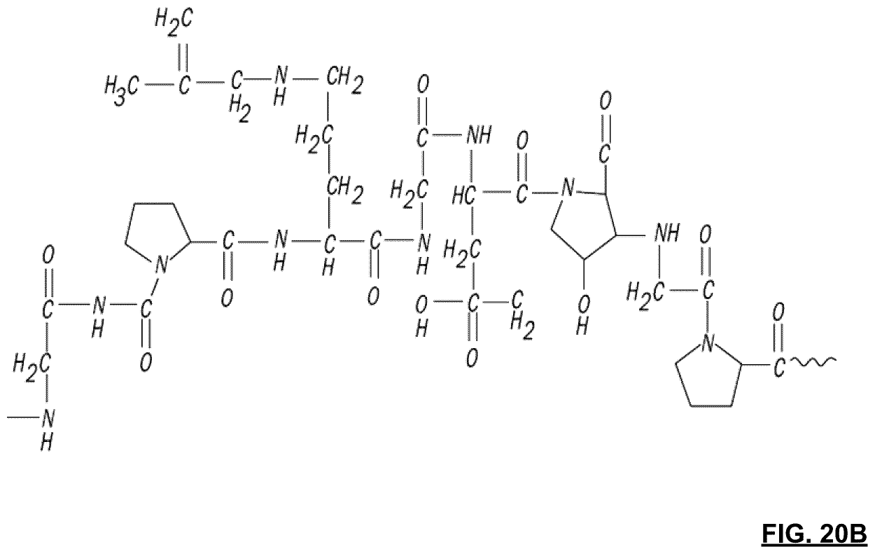

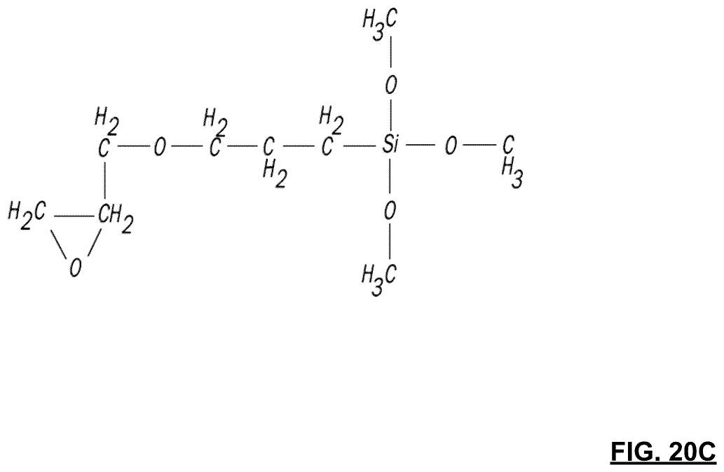

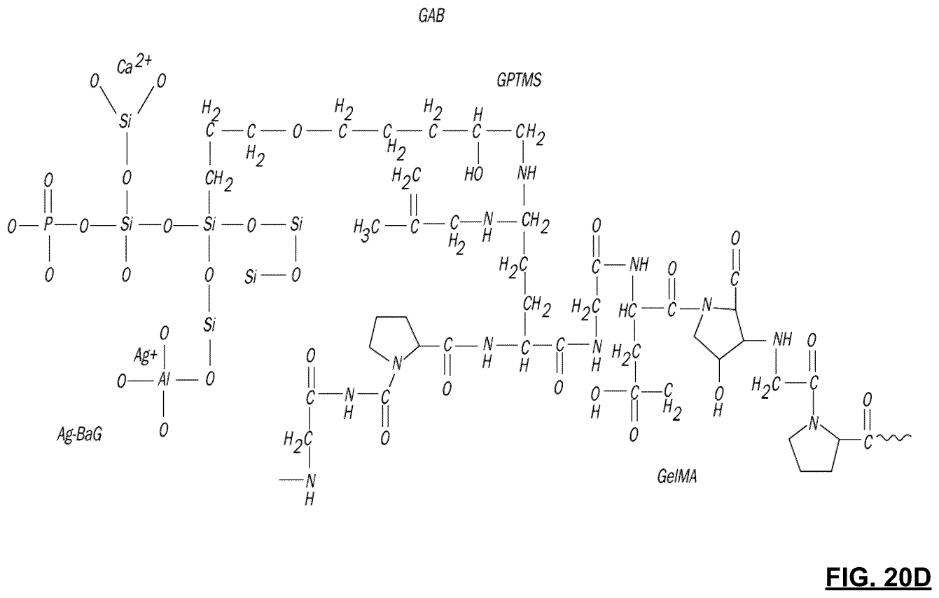

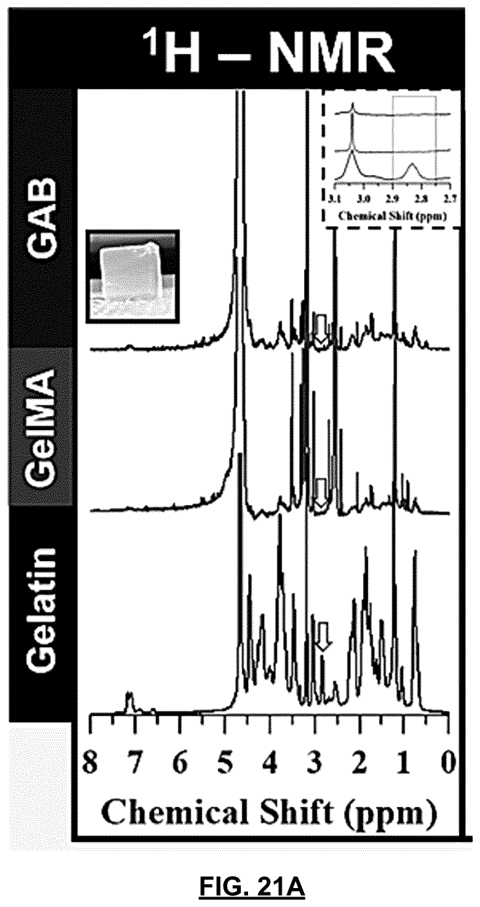

As shown in FIG. 20A, to synthesize the GAB hybrid material, the lyophilized GelMA (FIG. 20B) is dissolved at 10% (w/v) in DMSO at 50° C. and stirred at 500 RPM. A coupling agent, (3-Glycidyloxypropyl) trimethoxysilane (GPTMS; Millipore Sigma) (FIG. 20C), is then added to the solution to achieve a hydroxylysine, lysine, arginine to GPTMS ratio of 2.00 and allowed to react for 24h to ensure sufficient time for the epoxy ring-opening reaction, as shown in FIG. 20. The sol-gel process is used to synthesize the Ag-BaG following previously described methods. 3% (w/w) of the Ag-BaG sol is added to the GelMA solution in addition to distilled water to ensure sufficient hydrolysis between the GPTMS and Ag-BaG components and allowed to stir for 24h to achieve a homogenous distribution between GelMA and Ag-BaG at the molecular level. This solution is then precipitated, washed, and lyophilized as previously described to prevent the materials characteristics from changing during storage. Prior to further use, lyophilized GAB is dissolved at 10% (w/v) in 1× phosphate buffered saline (PBS) along with the photoinitiator VA-086 at 1.5% (w/v) and photopolymerized at 385 nm for 120 s producing GAB hydrogels (FIG. 20D).

### Structural Characterization

The overall morphological characteristics of the synthesized materials are evaluated using optical microscopy (VHX-600E Digital Microscope) and micro-computerized tomography (Micro-CT; Rigaku Quantum GX).

To study the microscopic morphological features of the synthesized materials, scanning electron microscopy (SEM; Tescan MIRA/JEOL 6610LV) is used. Samples are prepared for SEM examination by first undergoing a graded series of ethanol dehydration (i.e. 2×—25%, 2×—50%, 2×—75%, 2×—90%, and 3× 100% ethanol). The samples are then critically point dried using liquid CO₂ in order to preserve the native structure of the materials before being metalized with Os(g) for 15s to prevent a buildup of a negative electrical charge. Sample morphologies are captured using a beam voltage of 5 kV. Energy dispersive spectroscopy (EDS; Ametek EDAX Apollo X) is additionally performed to assess elemental homogeneity using a beam voltage of 20 kV.

To identify the molecular bonds present within the synthesized samples, Fourier-transformed infrared spectroscopy-attenuated total reflection (FTIR; Jasco FT/IR-4600) is applied, where spectra are collected from 4000-400 cm⁻¹. Additionally, spectra relating to the reagents used during the synthesis of GelMA and GAB are collected in order to utilize FTIR for quality control.

Proton nuclear magnetic resonance (¹H-NMR) is used to determine the methacrylation of gelatin yielded after performing the GelMA synthesis. The 1D spectra are collected using an Agilent DirectDrive2 500 MHZ NMR spectrometer, where a sample concentration of 50 mg mL⁻¹ in deuterium oxide (D₂O) is used for all measurements. The degree of substitution (DS) is calculated using the lysine integral method, where the integral of the lysine methylene signals of the gelatin as-received is compared to the integral of the lysine methylene signals in GelMA, as described in Eq. 3.

$${DS}_{Lysine} = ( 1 - \frac{\int Lysine GelMA}{\int Lysine Gelatin} ) * 1. \qquad Eq . 3$$

### Performance Characterization

To assess the swelling behavior of the synthesized samples, lyophilized samples are measured to quantify the mass of the lyophilized samples. The lyophilized samples are hydrated in 1×PBS forming samples 8 mm in diameter having a thickness of 1.5 mm and kept in solution for 24 h at 37° C. The hydrated samples are removed from the solution and excess PBS is gently blotted from the surface of the samples. The swelling ratio of the samples is calculated using Eq. 4.

$$\frac{( {W}_{t} - {W}_{0} )}{{W}_{0}} , \qquad Eq . 4$$

where Wₜ represents the weight of the swollen sample after 24h of immersion in PBS, and W₀ represents the weight of the lyophilized (dry) sample.

Samples are immersed in 1×PBS at 37° C. under shaking at 175 RPM to study their degradation behavior. After 1, 3, 5, 7, 14, 21, and 28d of immersion, the pH is measured, and extracts are collected at each time point to elucidate the ion release profile by inductively coupled plasma-optical emission spectrometer (ICP-OES). The samples at each time point are weighted to determine the mass loss profile and determine the time point at which complete degradation is expected.

The viscoelastic behavior of the samples is evaluated using rheological means. To this end, the storage (G′) and loss modulus (G″) of the samples is evaluated as a function of temperature ranging from ambient conditions to 40° C. The viscosity of the samples is evaluated at 37° C. using a shear rate from 0.1 to 100 s⁻¹.

The compressive behavior of the samples is evaluated using fully cross-linked samples. Samples 8 mm in diameter having a thickness of 1.5 mm re used and the compression testing is performed using a United SFM electromechanical series universal testing machine with a 20 N load cell. A constant cross-head speed of 0.5 mm s⁻¹ is used and all samples are compressed up to 60% strain. The elastic modulus is determined by the slope of the linear curve in the elastic region.

### Printability

The printability of the samples is investigated by varying the pressure (psi), needle gauge, extruder temperature (° C.), speed (mm s⁻¹), layer height (mm), cross-linking time(s), and cross-linking intensity (%) during 3D printing (Allevi 3). Semi-quantification of the printability is performed according to the previously reported procedures. In brief, the circularity of the printed enclosed area is calculated using Eq. 5:

$$C = \frac{4 π A}{{L}^{2}} , \qquad Eq . 5$$

where L represents the perimeter, A represents the area, and C represents the circularity, where a circularity of 1 represents a perfectly circular entity. Given the circularity for squared shapes is equal to ¼A, the printability parameter (Pᵣ), which is a quantification of similarity of the nature of square-formed prints, is calculated using Eq. 6:

$${P}_{r} = \frac{{L}^{2}}{16 A} . \qquad Eq . 6$$

The optimal or desired printability is achieved when Pᵣ=1 and denotes that the ideal viscosity during the print is achieved. It is important to note, as well, that when Pᵣ >1, the viscosity during the print is too high, and Pᵣ<1 denoting the viscosity is too low during the print.

### Results and Discussion

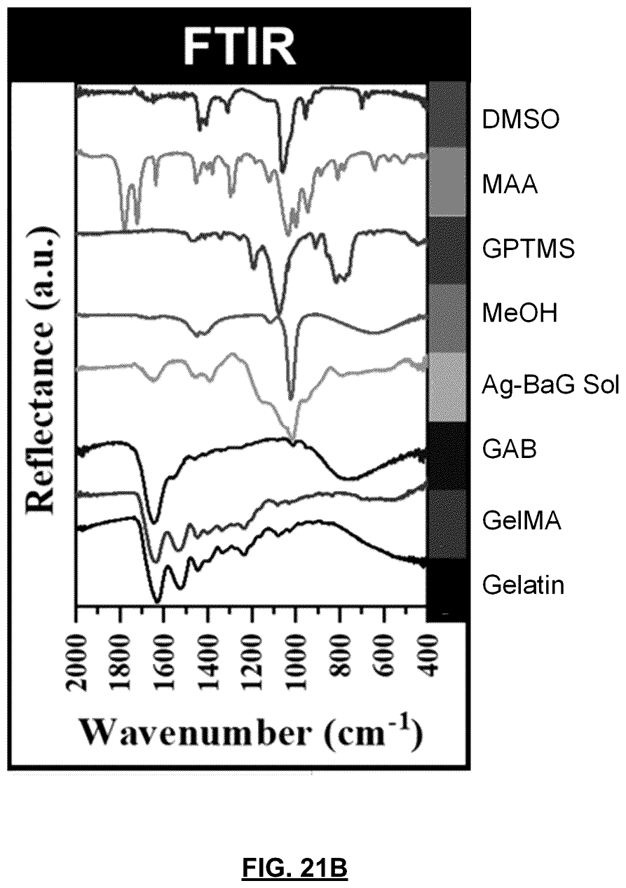

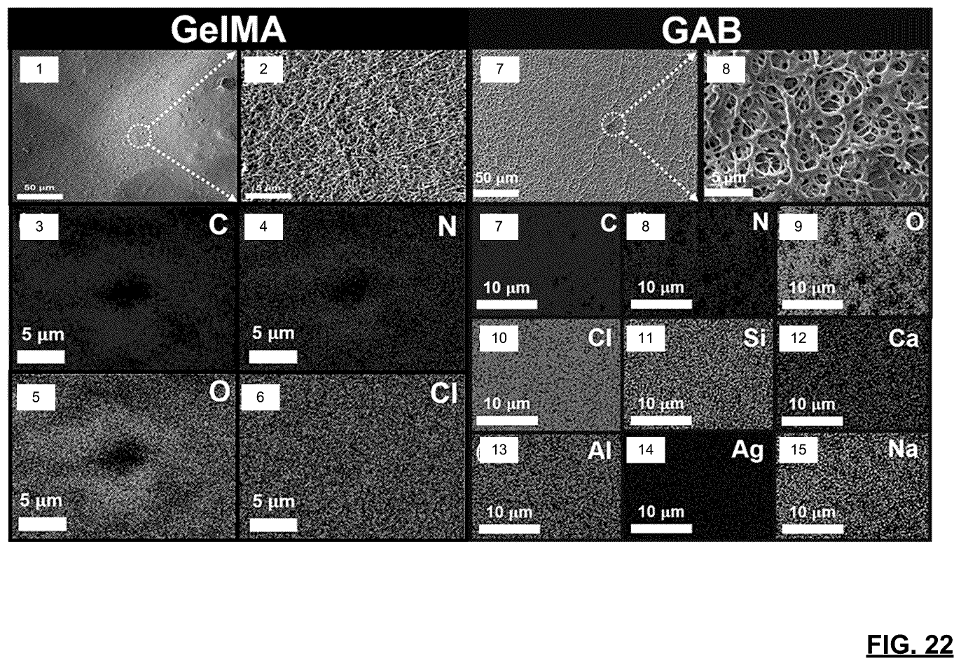

FIG. 21A shows the 1H-NMR spectra of the as-received gelatin, the synthesized GelMA, and the GAB. Using Eq. 3, the degree of substitution for GelMA and GAB using the lysine integral method is found to be about 100%, providing supporting evidence that the GAB synthesis does not compromise the degree of substitution.

Traditionally, GelMA has been synthesized in an aqueous environment such as PBS and using MAA as the methacrylation agent, leading to a maximum achievable DS of about 80 to about 85% with large batch-to-batch variations. This is believed to be a consequence of using an aqueous environment for the GelMA synthesis as the methacrylic anhydride preferentially hydrolyzes with its aqueous environment to form methacrylic acid, which is non-reactive with gelatin. To overcome or mitigate this and achieve a DS of about 80% to about 85% 10-32-fold molar excess of MAA should be used, compared to the lysine groups present in the gelatin. Lengthy dialysis (>7d) process is used to remove the remaining cytotoxic reagents (i.e. MAA). It has been demonstrated that the use of a carbonate-bicarbonate system tuned to have a pH of 9.0 found success in increasing the DS up to 97% while dramatically lowering the MAA required to a 2.2 molar excess, however, a lengthy dialysis post-processing step is still used.

Here, we aim to remove or reduce the adverse effects created by synthesizing GelMA in an aqueous environment through the use of an organic solvent, DMSO. It is hypothesized that the use of DMSO would prevent MAA hydrolysis and allow for about 100% DS to be achieved during synthesis. Furthermore, it was recently found that the dialysis step could be circumvented when using toluene as a precipitating agent. The small dielectric constant for toluene (ε of about 2.4), when exposed to the GelMA solution, increases the attractive forces between the oppositely charged portions of the GelMA allowing for agglomerations to grow resulting in the precipitation of the GelMA. An additional benefit to using the toluene precipitation method is that the DMSO and MAA are soluble in toluene allowing for their removal without the need for a dialysis process.

To verify the effectiveness of the toluene precipitation method, FTIR spectra (FIG. 21B) of gelatin as-received, GelMA, and GAB are collected in addition to the reagents used during the synthesis process of GelMA and GAB. As shown in FIG. 21B, minimal spectroscopic changes are observed after the synthesis of GelMA; an indication that the precipitation method used is successful at extracting the GelMA from the solution. Additionally, the characteristic spectroscopic peaks for MAA and DMSO are not observed in the FTIR spectrum of GelMA (FIG. 21B) providing further evidence that the precipitation method is successful. For the GAB, the FTIR spectrum (FIG. 21B) presented the characteristic amide I, amide II, and amide III groups at about 1650 cm⁻¹, about 1500 cm⁻¹, and about 1450 cm⁻¹ respectively demonstrating the GelMA structure is preserved during the GAB synthesis, as expected. The broad Si—O bending peak about 750 cm⁻¹ to about 800 cm⁻¹ is characteristic of the Ag-BaG indicative that there is successful incorporation and preservation of Ag-BaG during precipitation.

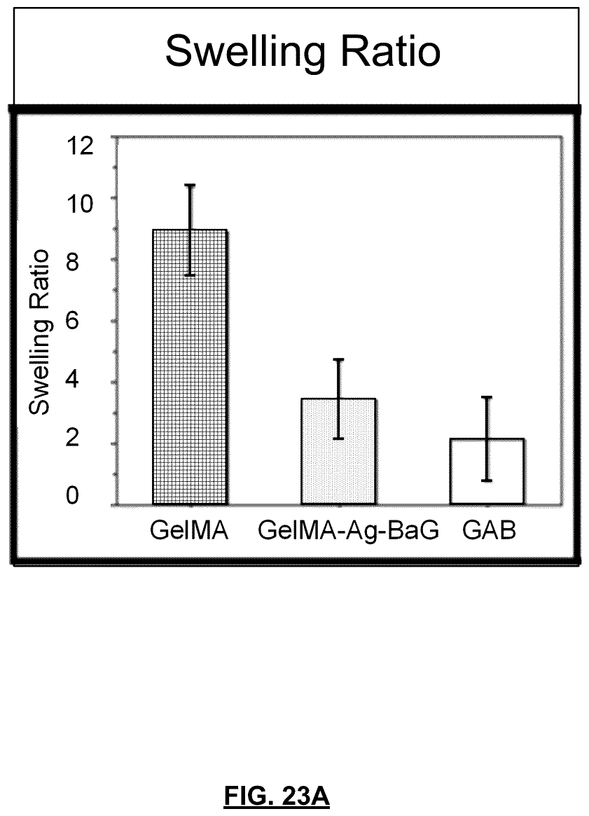

Given the successful synthesis of GelMA and GAB, when examining the morphology of both on the microscopic scale (FIG. 22 at 1, 2, 7, 8), clear morphological differences are observed. The GAB (FIG. 22 at 7, 8) show thicker filaments of material roughly one micron in thickness with relative uniformity likely a result of the combination of GelMA and Ag-BaG. Interestingly, the combination of the Ag-BaG with GelMA leads to a smoother surface morphology of the filaments compared to GelMA alone, where more node-like features are observed. The pore size of the GAB (FIG. 22 at 8) is additionally present on the micron-scale, whereas the pores for GelMA (FIG. 22 at 2) are evident on the sub-micron scale as a result of the sub-micron thick filaments.

Regarding elemental homogeneity, both GelMA (FIG. 22 at 3, 4, 5, 6) and GAB (FIG. 22 at 7, 8, 9, 10, 11, 12, 13, 14, 15) are found to have all elements homogenously distributed down to the micron level. This provides supporting evidence that the synthesis used to deliver GAB is additionally successful at achieving a high degree of homogeneity. This is important as this provides supporting evidence that GAB should give a homogenous response when studied in vivo; allowing for the benefits of the GelMA and Ag-BaG to be exhibited simultaneously.

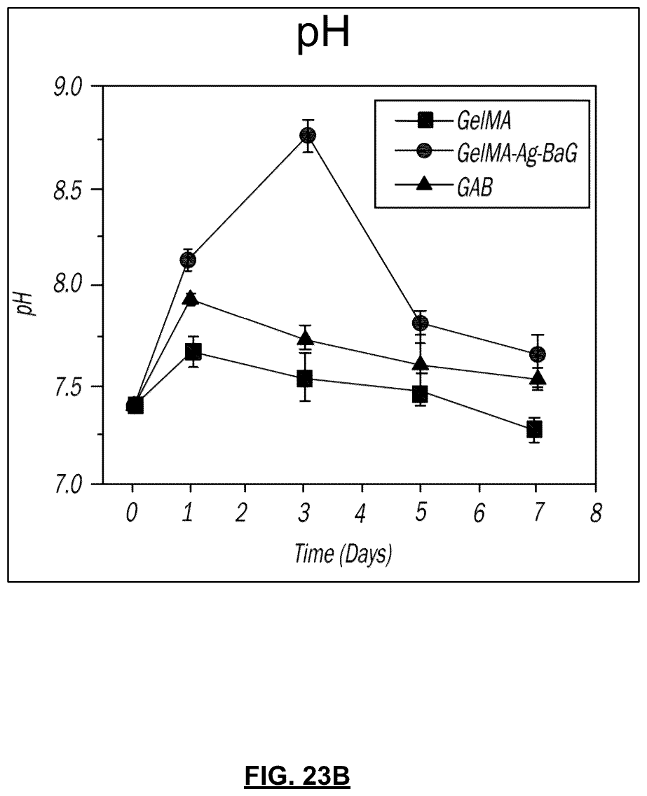

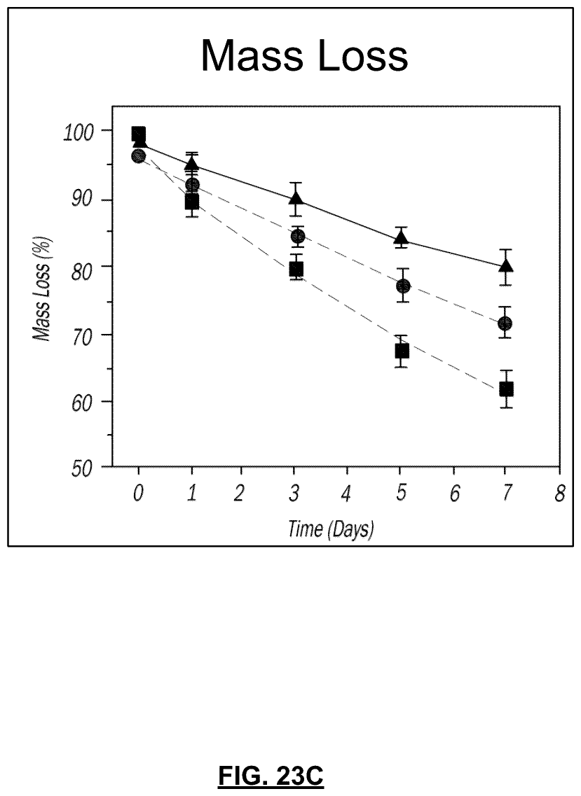

Regarding the performance aspects of GelMA and GAB in addition to the GelMA-Ag-BaG nanocomposite, the swelling ratio (FIG. 23A), pH evolution (FIG. 23B), and mass loss (FIG. 23C) are studied. It is found that the GAB undergoes the least amount of swelling compared to the GelMA or the nanocomposite, although the difference in the swelling ratio between the nanocomposite and GAB is insignificant. The presence of the Ag-BaG in both the nanocomposite and GAB decreases the ability of the material to swell given the Ag-BaG does not experience swelling when exposed to aqueous environments. The swelling ratio (FIG. 23A) for GAB is likely decreased compared to the nanocomposite as a result of the covalent bonding that existed between the Ag-BaG and GelMA as opposed to the weaker Van der Waals interactions between Ag-BaG NPs and GelMA.

The pH evolution over the course of seven days (FIG. 23B), reveals that GAB presents an evolution that models, well, the pH evolution of GelMA. This is likely due to the covalent bonding present in addition to the fine degree of homogeneity achieved during the synthesis process allowing the GelMA to mediate the ion release from the Ag-BaG compared to the nanocomposite. The GelMA-Ag-BaG nanocomposite displays a large increase in pH that peaked at about 8.8 before dropping below a pH of 8 after 5d of immersion. Sol-gel-derived bioactive glasses have a burst release of ions at early time points of immersion creating an alkaline environment, as evidenced in FIG. 23B. This presents supporting evidence of the benefits that can be achieved when combining the GelMA with the Ag-BaG to create the hybrid material. Additionally, the GAB is found to be the most stable when the mass loss (FIG. 23C) is studied up to seven days compared to the nanocomposite, demonstrating further the advantages of covalently bonding the GelMA with the Ag-BaG.

To determine whether GAB is a viable material for 3D printing, its rheological performance is studied. Table 3 shows a summary of the viscosity of GAB as a function of shear rate in addition to the storage and loss modulus and compared to values for GelMA obtained from literature along with the recommended values for each materials property for targeting 3D printing applications.

TABLE 3
Experimental and reported values for the rheological
properties of GelMA-containing bioinks.

| | GAB Bioink | | | GelMA Bioink | | | Recommended |
|---|---|---|---|---|---|---|---|
| | 10% (w/v) | | | 10% (w/v) | | | Values from |
| Property | GelMA | | | Literature | | | Literature |
| Viscosity (Pa · s) | 40,000 | ~1,000 | ~50 | ~300 | ~170 | ~40 | 0.03-60,000 |
| Shear Rate (s⁻¹ | 0.1 | 1 | 10 | 0.1 | 1 | 10 | — |
| Storage Modulus | ~75,000 | | | ~800-1,000 | | | 100-1,000 |
| (Pa) | | | | | | | |
| Loss Modulus | ~20,000 | | | ~5-40 | | | — |
| (Pa) | | | | | | | |

| | | | |
|---|---|---|---|
| Shear Thinning | ✓✓✓ | ✓✓✓ | ✓✓✓ |

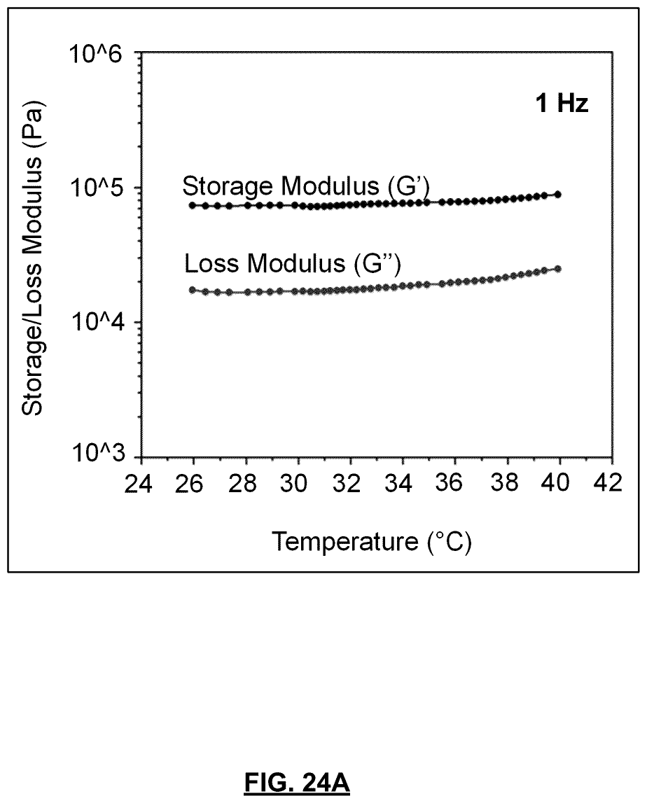

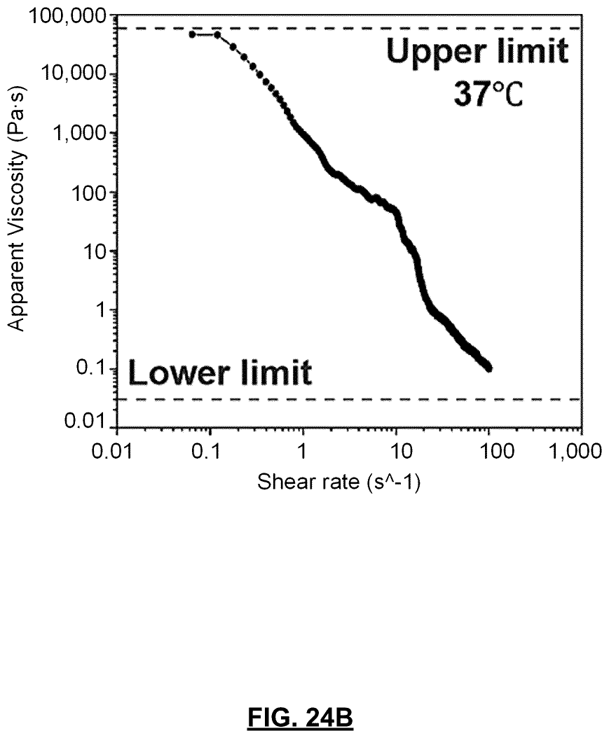

For viscoelastic materials such as GelMA and GAB, the storage modulus (FIG. 24A) represents the ability of the material to absorb deformation energy elastically, whereas the loss modulus (FIG. 24A) represents the energy dissipated when a force is removed. For GAB, the storage modulus is found to be two orders of magnitude greater than either the GelMA reported in the literature or the recommended values (Table 3). The covalent coupling between GelMA and Ag-BaG allows the material to absorb a much greater amount of deformation energy elastically by providing avenues where the strong ionic bonds from the Ag-BaG can contribute to the deformation energy storage. The loss modulus for the GAB is expectedly increased compared to GelMA given the presence of the Ag-BaG naturally increases the stiffness as a result of the orders of magnitude higher stiffness for bioactive glasses such as Ag-BaG compared to a softer material, such as GelMA.

The viscosity of the GAB (FIG. 24B) is found to decrease as a function of increasing shear rate, an indication of shear-thinning behavior. It is preferential for the GAB to exhibit shear-thinning behavior as shear thinning allows materials to exhibit self-healing abilities after removal of the shear forces, allowing for higher quality constructs to be 3D printed. The linear region present between about 1 s⁻¹ to 10 s⁻¹ (FIG. 24B) is used to determine the flow consistency index (K) and flow behavior index (n) using a power fitting performed following Eq. 7.

$${η}_{app} = K {\dot{γ}}^{( n - 1 )} . \qquad Eq . 7$$

From Eq. 7, GAB is found to have a flow consistency index (K) of 417±10 (Pa·sn) and a flow index behavior of 0.045±0.007 with a correlation coefficient (R²) of 0.98. Given the low value of n for GAB, this is further supporting evidence to suggest that GAB exhibits shear thinning behavior and furthermore behaves as a non-Newtonian fluid.

FIG. 24C shows a single-layer mesh 3D printed using GAB, where GAB is successfully printed using a pressure of 15 psi, an extruder temperature of 27° C., a 27 gauge metal tapered-tip, and a printing velocity of 4 mm s⁻¹. The ability to 3D print GAB at 15 psi with an extruder temperature only 10° C. cooler than physiological temperatures should reduce or minimize the shear forces and reduce or minimize the temperature differential cells would exhibit when incorporated into the material for 3D bioprinting. This presents supporting evidence of the printability of GAB and demonstrates its potential advantages for 3D bioprinting compared to other hydrogels such as GelMA. Furthermore, the ability to successfully incorporate Ag-BaG into GelMA without requiring the use of NPs is expected to improve cell viability during 3D printing given the introduction of additional shear forces due to the presence of the NPs.

### Conclusion

Following this approach, chemically bonded GelMA-Ag-BaG (GAB) is successfully synthesized and cross-linkable enabling 3DP. The rheological evaluation shows that the GAB exhibited shear thinning behavior, which is a preferential characteristic for printability. The incorporation of the Ag-BaG is found to be homogenous at the molecular level that leads the GAB to exhibit the least amount of swelling and the slowest degradation behavior compared to either the GelMA alone or the GelMA-Ag-BaG NP nanocomposites. GAB is expected to be suitable for extrusion-based 3DBP technologies and expected to improve cell viability of the 3D printed constructs as a result of the improvements in the characteristics of the material of the GAB bioink over a GelMA-Ag-BaG NP nanocomposite bioink.

### Example 4

This example describes an oxygenated osteopromotive bioink that preserves cell viability extrusion-based 3D bioprinting.

### Summary

Bone tissue loss can occur during surgery, and large bone defects can be caused by congenital malformation, trauma, infection, or cancer. Treatments can include use of allografts, biomaterials, or autografts. Recent development of 3DBP technology facilitates the development of customized bone substitutes. Bioinks used for bone 3DBP employ various particulate phases such as ceramic and bioactive glass particles embedded in the bioink. When such bioinks used with the most commonly used 3DBP method of extrusion-based 3DBP, cells are exposed to possible damage due to in homogenously distributed particles. Therefore, the objective of this example is to develop cell friendly osteopromotive bioink. To achieve this, the example links GelMA and Ag-BaG to produce a hybrid GAB material. The distribution of glass elements in the resulting hybrid GAB structure is examined. Rheological properties of the resulting hydrogel and its printability as well as degree of swelling and degradation over time are also evaluated. GaB is compared to GelMA and GelMA/Ag-BaG blends. Results show the superiority of chemically complexed GAB bioink in terms of homogenous distribution of the elements in the structure, rheological properties, printability and degradation profiles. Accordingly, such a new bioink represents a major advance in bioinks for bone 3DBP.

### Introduction

Large bone defects can be caused by congenital malformations, trauma, infection, cancer or surgical resection and represent a major clinical challenge in orthopedic and CMF surgery. An important approach in the treatment of these defects is to use autografts, but allografts or biomaterials are also employed. There are about 2 million bone graft procedures performed annually worldwide to treat bone lesions. Autografting is limited by availability, donor-site morbidity and the difficulty to adapt to the required shape, for example, in the CMF area. The use of devitalized allografts is costly and employs a laborious process, and resulting repairs have limited revascularization with significant risks of immunological reaction and infection. Infected bone often undergoes secondary revision surgery, which prologs recovery time and may result in permanent functional loss. It would therefore be beneficial to develop effective tissue engineering approaches that can regenerate the damaged tissue and also combat infection.

Biomaterials contain no living cells, and they elicit a foreign body reaction which leads to osteolysis. To make biomaterial-based approaches more biomimetic, it is possible to seed on polymers to form tissue engineering constructs, and these have been evaluated in the body. Unfortunately, many synthetic biomaterials are not cell-friendly. Therefore, natural materials such as decellularized tissue matrices, or polymers such as collagen and gelatin, which possess the RGD peptide and are important for cell attachment and function, have been used either alone or in combination with synthetic materials and growth factors as alternative materials. Although these constitute an important advance in the field of tissue engineering, inclusion of cells appears to also be important, and cell seeding has not proven to be the solution since it leads to inhomogeneous cell distribution and subsequent failure of implants.

The advent of the 3DP technology facilitates the development of customized bone substitutes, aided by images derived via CT, MRI, and CAD. The adaptation of 3DP to produce cell-laden constructs (3DBP) has led to advances in engineering of biomimetic living constructs. 3DBP patient-specific constructs can contribute significantly to improved treatment outcomes and accelerated recoveries. 3DBP is expected to revolutionize the biomedical engineering field.

The search for alternative methods led to the adoption of the technique of 3DP into 3DBP by adding cells to matrix material present in liquid form and its subsequent solidification following printing into desired form tissue constructs.

The most commonly used 3DBP method relies on extrusion of cell-laden bioinks, which unfortunately is associated with reduced cell viability as compared to other methods of 3DBP. This is possibly due to the mechanical shear stress. It is hypothesized that this effect is aggravated with the addition of solid elements such as ceramics or glass to the bioink.

Current bioinks used for bone 3DBP usually include osteopromotive elements in the form or calcium-based ceramics or silicate-based materials such as BaG, commonly in the form of particles (micro- or NPs). They are usually blended with a matrix polymer in which cells are embedded to form a bioink, which promotes both angio- and osteogenesis. There are examples where the NPs of silicate are combined with the polymer GelMA and ionic interaction keeps the two components together. Conventional composite bioinks cannot ensure homogeneous distribution of osteoconductive particles and even may lead to agglomeration and damage to cells during the printing process, most commonly accomplished using extrusion bioprinting, which is associated with shear stress. Recently, it is found that addition of BaG particles to matrices lead to death of osteoblasts.

It is therefore important to develop cell-friendly osteopromotive bioink. It follows that, it would be beneficial to address these problems by having the matrix component and the osteopromotive component chemically linked. Chemical bonding of both matrix and osteopromotive element will result in a stable structure and prevent unwanted effects and limitations of conventional osteogenic bioinks.

Such materials that combine these components and properties have not been previous reported and these advances in materials for 3D bioprinting could have a major impact on 3DBP of bone tissue in terms of cell viability, osteogenic differentiation, angiogenesis, antibacterial properties and cell survival in vivo (under hypoxic conditions before new blood vessels form).

In addition to its role in bone regeneration, BaG has also anti-inflammatory and pro-angiogenic properties. Hence, BaG component will provide multiple functions including: 1) inducing optimal or improved osteogenic response in cells due to BaG itself and ions that are released following its dissolution, 2) enhancing mechanical properties of the composite scaffolds, 3) possessing anti-inflammatory properties and 4) promoting angiogenesis. Angiogenesis and osteogenesis are known to go hand-in-hand in normal bone healing, and therefore this may be of great importance for the success of any bone regenerative bioink. 3DBP is expected to revolutionize the biomedical engineering field, and creation of innovative bioinks that support cell growth and differentiation lie at the center of this emerging technology.

To successfully engineer durable bone tissue constructs, a radically innovative approach is required. The innovative aspects of our novel GAB hybrid hydrogel are based on the unique antibacterial and regenerative composition that mimics native bone tissue. The innovations in our material and approach include: 1) an organic component in the form of collagen-derived gelatin for enhancing cell attachment and function; this is linked to methacryloyl to facilitate crosslinking the polymer (GelMA); 2) an inorganic component in the form of new composition of Ag-doped BaG, which has osteogenic, angiogenic and antibacterial properties, and enhances mechanical properties of the material; 3) the two components (Ag containing BaG and GelMA) are chemically linked to form a hybrid material, GAB; Although, BaG particles (with poor antibacterial activity) had been added to GelMA to produce inks for 3D printing in composite form (blend) or as a hybrid (not used for 3DBP; limited to 3DP only), the present chemically linked version has superior properties.

### Materials and Methods

### Material Synthesis

GelMA is synthesized GelMA. Briefly, type A gelatin is dissolved in DMSO. MAA is added to achieve a MAA:lysine. Toluene is added and left to precipitate out the GelMA, which is washed, dried, immersed in distilled water, vortexed, centrifuged, and supernatant discarded. GelMA is dissolved in distilled water before lyophilization and storge.

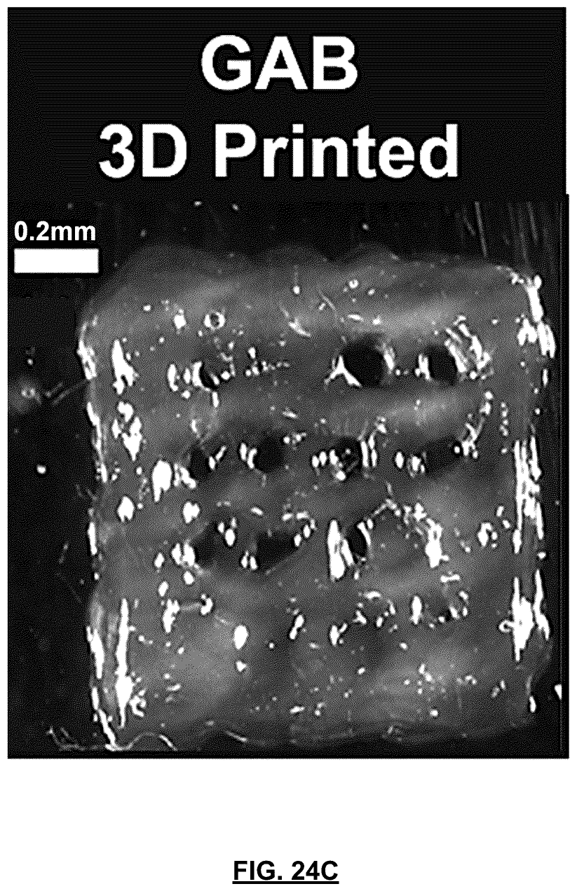

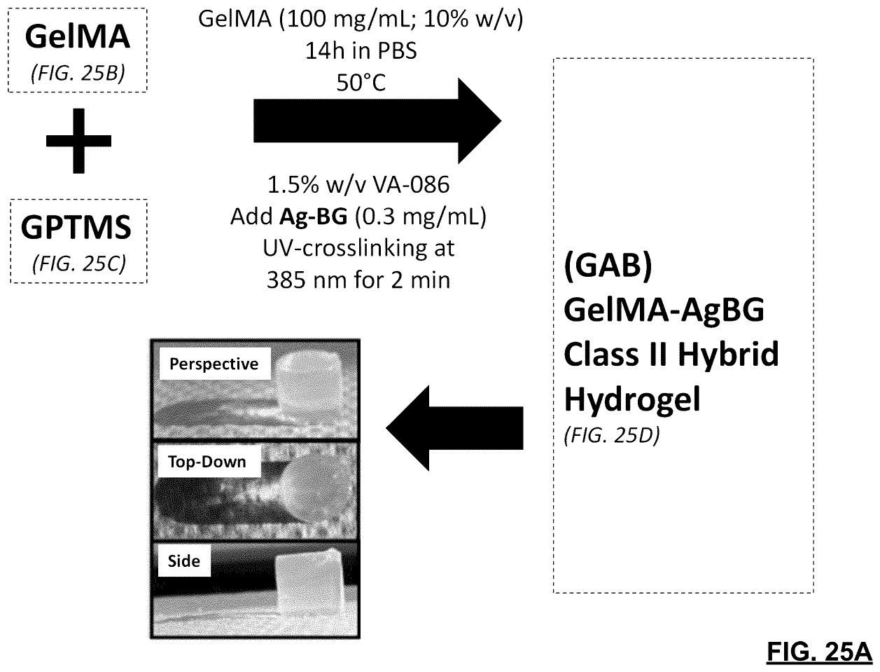

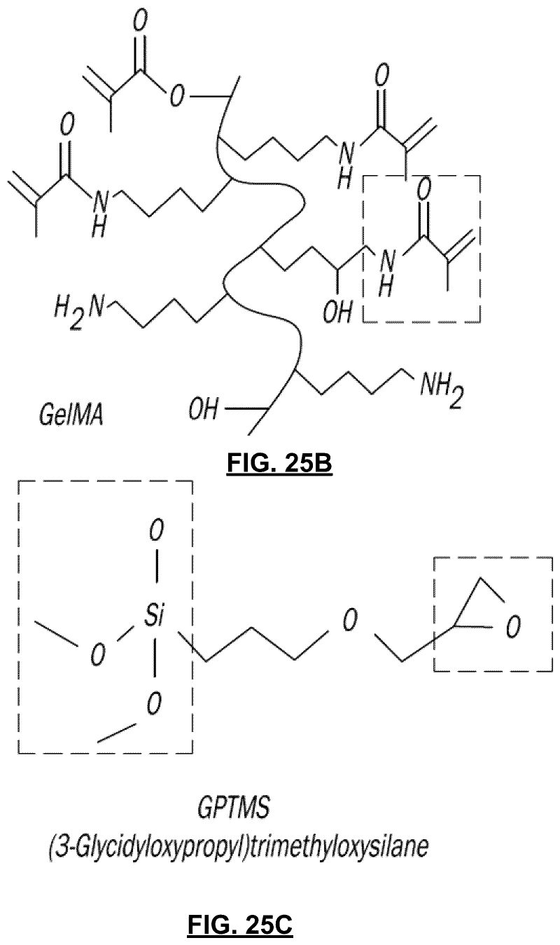

As shown in FIGS. 25A-25D, for the synthesis of GAB hybrid material, GelMA (FIG. 25A) is dissolved at 10% (w/v) in PBS at 50° C. GPTMS (FIG. 25B) is then added to achieve a hydroxylysine, lysine, arginine to GPTMS ratio of 2.00 and reacted with the dissolved GelMA at 40° C. for 14h to allow for sufficient epoxy-ring opening. Sol-gel process is used to synthesize the Ag-BaG. Then, 3% (w/w) of Ag-BaG solution is added to the dissolved GelMA at 40° C. and allowed to stir for 2h to achieve a homogenous distribution between the GelMA and Ag-BaG at the molecular level. A photoinitiator (VA-086) is dissolved into the GAB solution at a concentration of 1.5% (w/v), GAB is photopolymerized at 385 nm for 2 min. and stored at 4° C. until needed.

### Characterization: Morphological, Structural and Chemical, Characterization

The microstructure of samples is studied by using SEM. Microstructure and porosity of the hydrogels are analyzed using ImageJ software and SEM images. Also, energy disperse X-ray (EDX) spectroscopy is used for elemental analysis and homogeneity.

To determine molecular bonds present, identify GelMA and Ag-BaG components within spectra, and carry out quality control for consistency and purity, FTIR is used.

NMR is used to determine degree of substitution of GelMA and to assess bonding between GelMA-GPTMS and GelMA-Ag-BaG-GPTMS.

Swelling studies are carried out to determine the swelling ratio by comparing dry weight (lyophilized) to weight after immersion into DPBS solution and kept at 37° C. for 24 h (8 mm diameter and 1.5 mm thickness). The weight of swollen hydrogels is defined after removing excess water from the surface. The swelling ratio of hydrogels is calculated according to the formula: (Wₜ−W₀)/W₀; where, Wₜ and W₀ represent the weight values of fully swollen and lyophilized hydrogels, respectively.

Degradation studies are carried out by immersing hydrogels in medium at 37° C. under shaking, collecting extracts at desired time points of 3, 6, 9, 14, 21, and 28 days. Changes in the weight and the ion release profile are monitored. To evaluate morphological changes, imaging characterization is performed using μCT, on all samples at each time point.

### Characterization: Mechanical Properties

For rheology, storage/loss modulus (Pa) as a function of temperature (RT→40° C.), and viscosity as a function of shear rate are evaluated. A solvent trap is used to prevent dehydration. The temperature dependence of G′ and G″ is obtained using temperature sweep (oscillation) by decreasing the temperature from 24° C. to 4° C. at a cooling rate of 1° C. min-1. Then, the viscosity of bioinks is recorded at 4° C. from 0.1 to 100 shear rate.

For testing compression, hydrogels are cast in polydimethylsiloxane (PDMS) molds (8 mm diameter and 1 mm height) and exposed to the UV light (4 W cm⁻²) for 40 s for crosslinking, and evaluated at a cross speed of 0.5 mm s⁻¹ and up to 60% strain, using a mechanical testing machine (Instron).

The Young's modulus is determined based on the slope of the initial linear region, corresponding to 10% strain.

### Characterization: Printability

Printability under various conditions (including pressure, needle gauge, ambient temp, UV (intensity and duration) is investigated. Semi-quantification of bioink printability is performed according to known procedure, where printability parameter (Pᵣ), is used to quantify similarity with the nature of square-formed prints is defined. It is based on the circularity of enclosed area, which is defined as C=4πAL−2, where L is perimeter and A represents area. The highest circularity is at the C=1 condition. When the C value is closer to 1, the shape is close to the circle. The circularity for squared shapes is equal to α/4 rather than circles and the Pᵣ defined as Pᵣ=πC−¼=L2A− 1/16. For optimal or desired printability, Pᵣ should be 1, when the connected channel forms a complete square shape at ideal viscosity of bioink. Higher Pᵣ is obtained with higher viscosity, and lower Pᵣ with lower viscosity. In Image-J program, optical images of printed constructs are analyzed to determine the perimeter and region of the interconnected channels to calculate the Pᵣ of each printed construct (n=5).

### Results and Discussion

### BaG is Homogeneously Distributed in the Hydrogel Structure

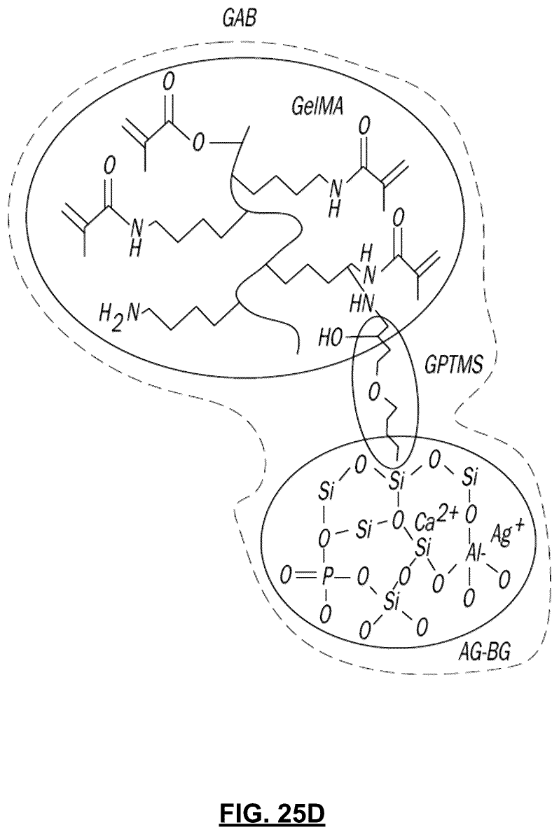

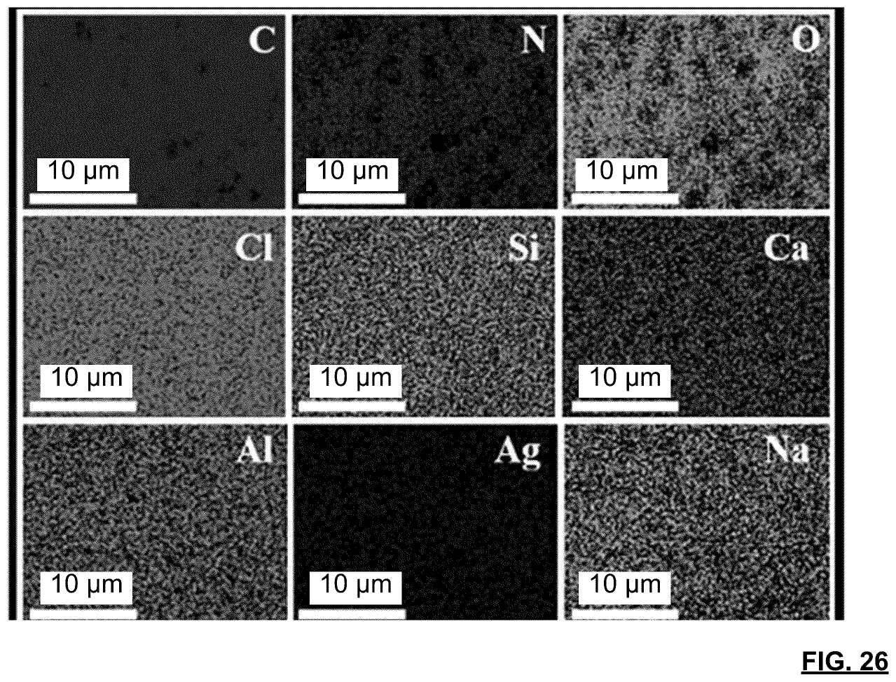

Homogeneous distribution of various elements in the GAB material is demonstrated (FIG. 26).

GaB is Highly Porous but Swells Less than Corresponding Blend and GelMA

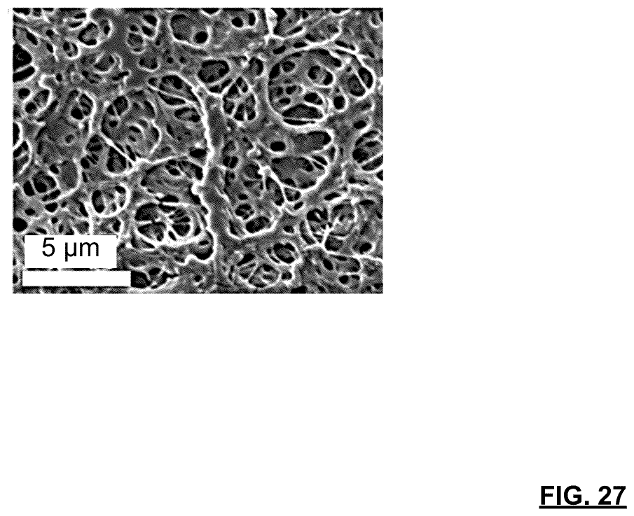

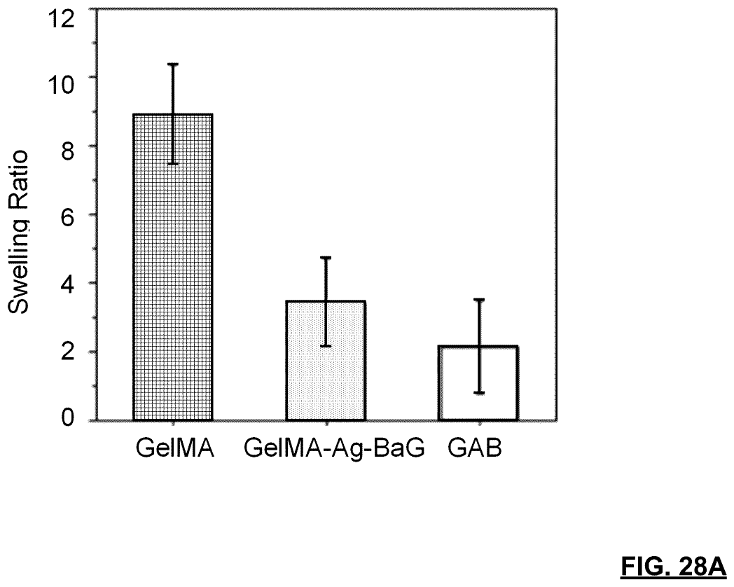

Highly porous structure of GAB material on the microscale is demonstrated (FIG. 27). GAB undergoes less swelling as compared to GelMA and blend materials (FIG. 28A) and it is also more stable losing mass at slower rate as compared to them (FIG. 28B).

### GaB Material is Appropriate Rheological Properties Suitable for Printability

Storage modulus is determined to be ˜about 2 orders of magnitude greater than what literature recommends. The viscosity of the GAB Class II hybrid hydrogel is within an acceptable range given the applied shear rate is <100 s⁻¹. There is a decrease in viscosity as a function of shear rate indicating shear thinning behavior for GAB Class II hybrid hydrogel (Table 4).

TABLE 4
Experimental and literature values for rheological properties of GelMA bioinks.

| | GelMA-AgBaG | | | GelMA Bioink | | | | |
|---|---|---|---|---|---|---|---|---|
| | Bioink | | | (10% w/v) | | | Recommended | |
| Property | (10% w/v GelMA) | | | Literature | | | Literature | |
| Viscosity (Pa · s) | 40,000 | ~1,000 | ~50 | ~300^{[5]} | ~170 | ~40 | 0.03-60,000 | [1-2] |
| | | | | | [5] | [5] | | |

| | | | | | | | |
|---|---|---|---|---|---|---|---|
| Shear Rate (s\hat{ }−1) | 0.1 | 1 | 10 | 0.1 | 1 | 10 | — |
| | | | | [5] | [5] | [5] | |

| Storage Modulus | ~75,000 | ~1,000 | [6] | 100-1000 | [1-2] |
|---|---|---|---|---|---|
| (Pa) | | ~800 | [7] | | |
| Loss Modulus | ~20,000 | ~5 | [6] | — | |
| (Pa) | | ~40 | [7] | | |

| | | | |
|---|---|---|---|
| Shear Thinning | ✓✓✓ | ✓✓✓ | ✓✓✓ |

This behavior indicates that hydrogel will exhibit self-healing abilities after removal of shear forces. Power fitting can be used within the range of about 1-10 s⁻¹ to determine flow consistency index (K) and flow behavior index (n)

$${η}_{app} = K {\dot{γ}}^{( n - 1 )} , \qquad Eq . 8$$

where K=417±10 (Pa·sn), n=0.045±0.007, R²=0.98.

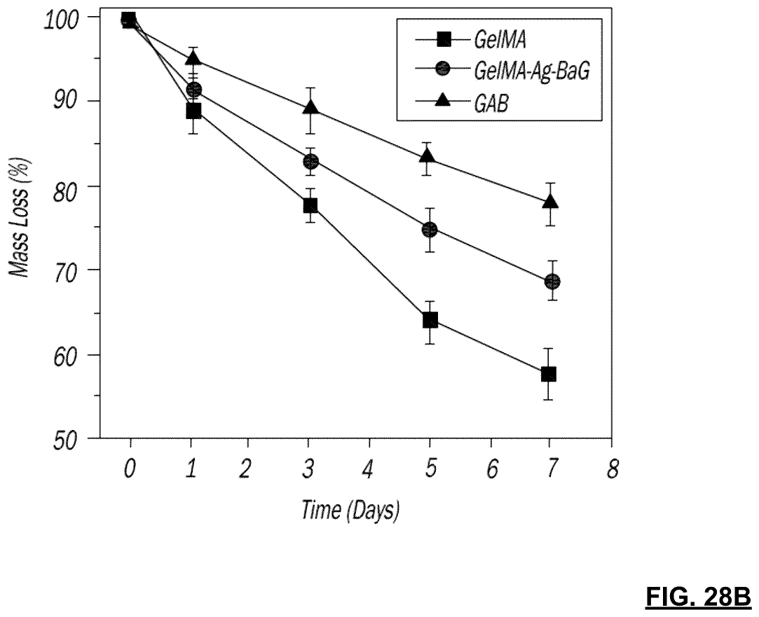

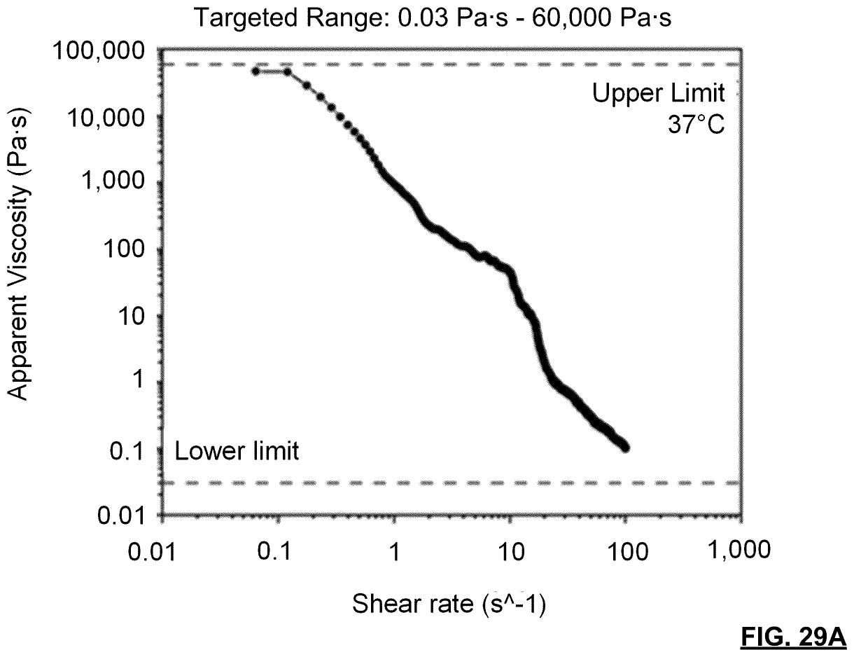

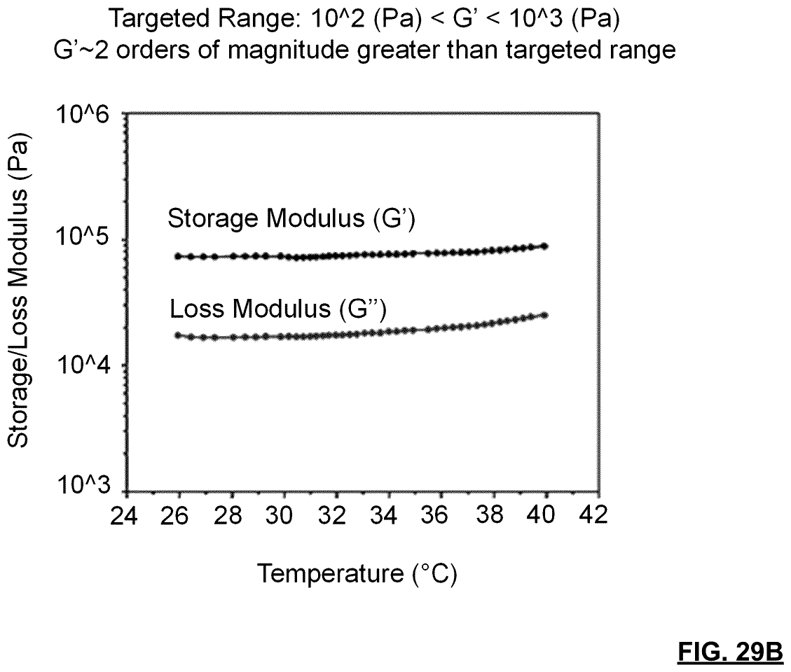

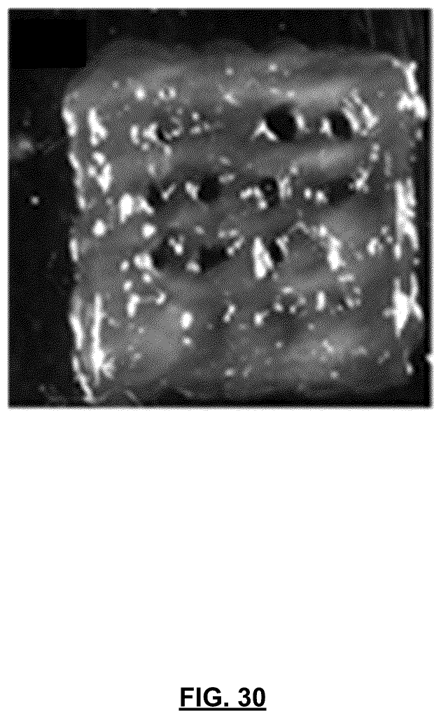

The flow behavior index (n) indicates that the GAB hydrogel is highly shear thinning (i.e. non-Newtonian fluid, pseudoplastic) (FIG. 29A-29B). GAB printability into a mesh is also demonstrated (FIG. 30).

### GaB Material has Bactericidal Effect on MARSA

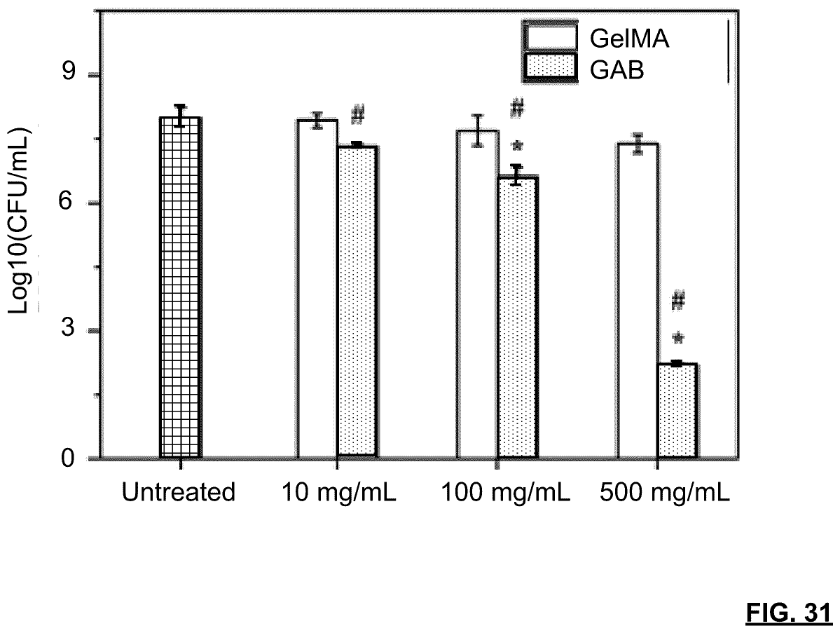

FIG. 31 shows inhibition of bacterial growth (*) p<0.05 comparing samples within the same time point, (#) p<0.05 comparing the same sample at different time points.

### GaB Material is Cytocompatible and Allows Fibroblast Proliferation

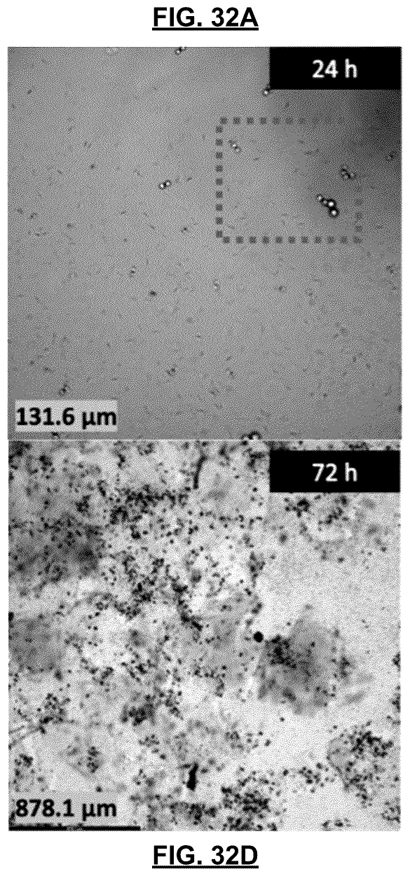

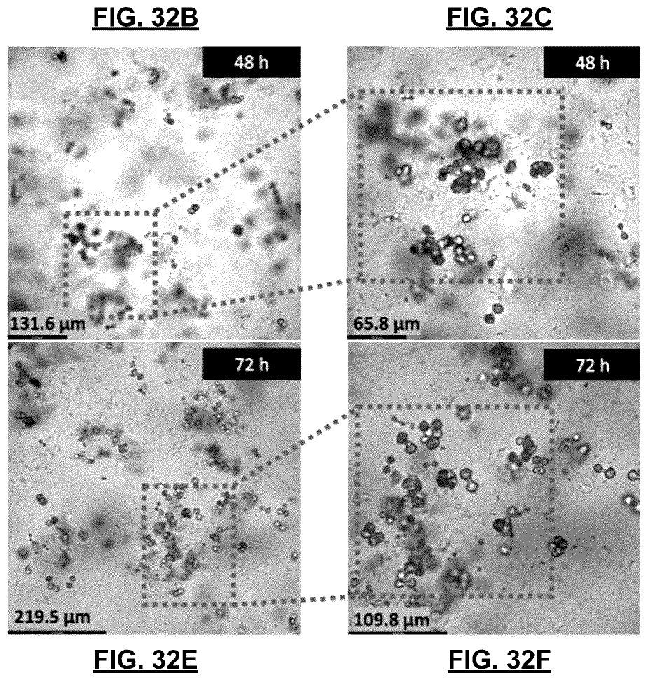

As shown in FIGS. 32A-32F, 3T3 fibroblasts are added to the GAB hydrogel, crosslinked, and incubated. No cytotoxicity is observed. Rather cells showed excellent proliferation over 24-72 h. Fibroblasts grow promptly even after 10 mins UV crosslinking with 385 nm. They keep growing in all 3D directions. More homogenous mixing with cells is possible in GAB hydrogel. Cells look alive and proliferate all over the scaffold. No cytotoxicity is observed. Rather cells showed excellent proliferation over 24-72 h. Fibroblasts grow promptly even after 10 mins UV crosslinking with 385 nm. They keep growing in all 3D directions. More homogenous mixing with cells is possible in GAB hydrogel. Cells appear to be alive and proliferating all over the scaffold.

GaB Material Allows Cell Loading and Extrusion of Fibroblasts and MSCs with Higher Viability as Compared to Corresponding Blends

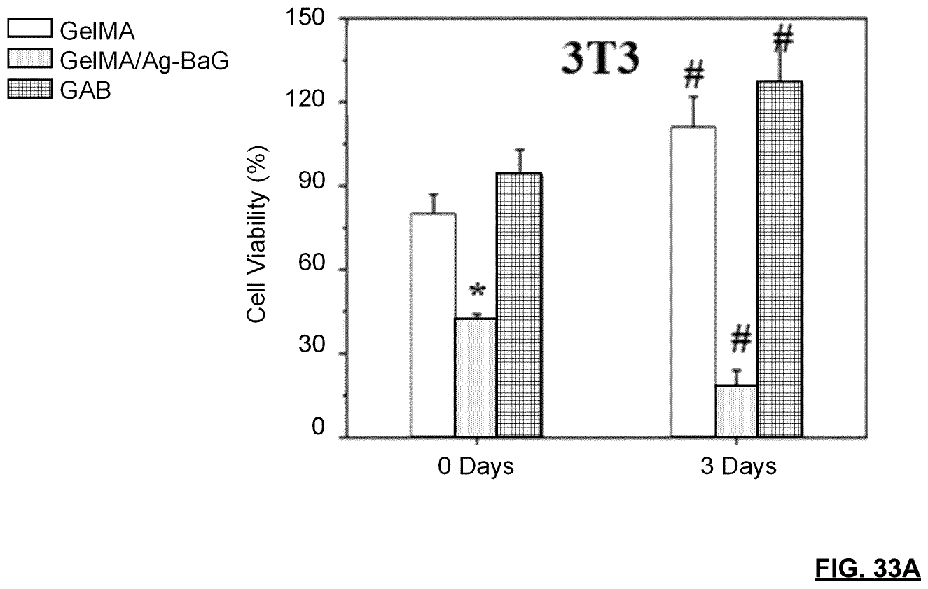

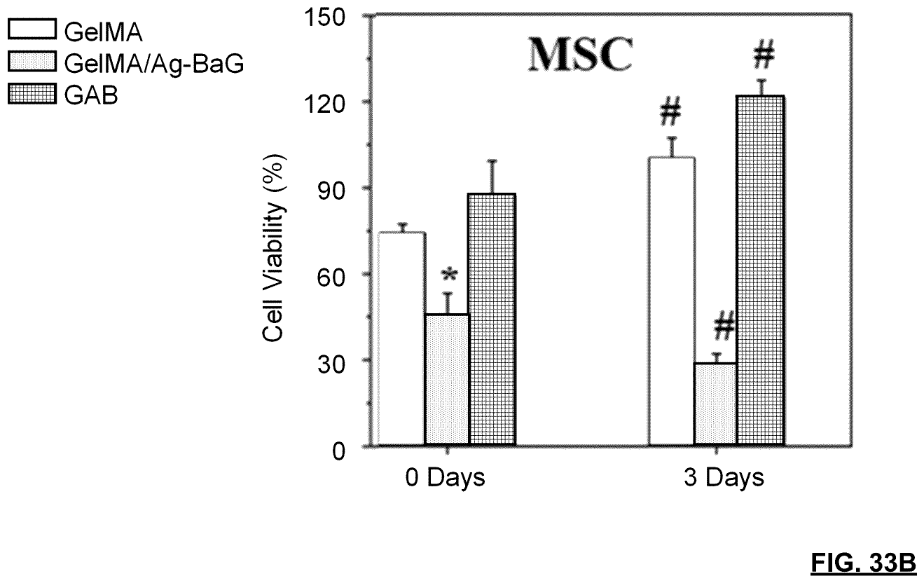

FIGS. 33A-33B illustrate metabolic activity of MSCs and fibroblasts in extruded 3D GelMA, GAB and blend constructs. (*) p<0.05 comparing samples within the same time point and (#) p<0.05 comparing the same sample at the two different time points.

The Addition of CPO to GaB Material Maintains Viability after Extrusion of Fibroblasts and MSCs Under Hypoxic Condictions as Compared to Corresponding Blends

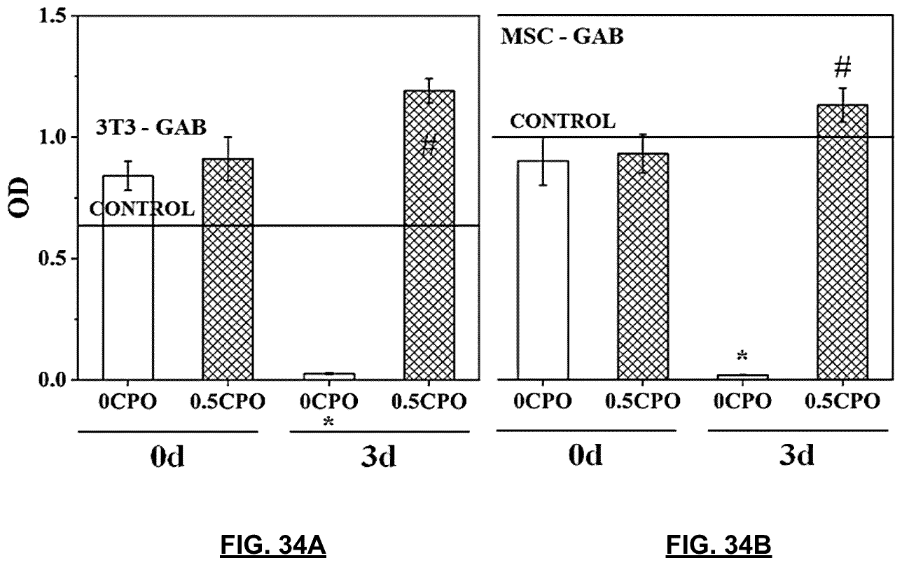

FIGS. 34A-34B depict O₂ release from CPO embedded in GelMA hydrogel in different concentrations.

### CONCLUSION

It is feasible to produce hybrids of gelatin methacryloyl and silver bioactive glass that are formed by chemically linking both comments to form a shear thing material which is crosslinkable to facilitate 3D printing. This way homogeneous distribution of the additive glass in the resulting hybrid material is ensured. It is also found that this new material undergoes less swelling as compared to blends of these elements or of GelMA alone. In addition the hybrid is comparatively more stable over time. The material can be suitable for the 3DBP using extrusion methods which are anticipated to improve the viability of cells in the printed constructs for the first time used improved bioink properties.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A biomaterial comprising:
gelatin methacryloyl (GelMA); and
a silver-containing bioactive glass (AgBaG) moiety,
wherein the AgBaG moiety is chemically attached to the GelMA to define GelMA-AgBaG.

2. The biomaterial according to claim 1, wherein the AgBaG moiety is chemically attached to the GelMA by way of a covalent bond.

3. The biomaterial according to claim 1, wherein the AgBaG moiety is chemically attached to the GelMA by way of a linker.

4. The biomaterial according to claim 3, wherein the linker is (3-glycidyloxypropyl) trimethoxysilane (GPTMS).

5. The biomaterial according to claim 1, wherein the AgBaG moiety comprises oxides of silicon, calcium, and phosphorus.

6. The biomaterial according to claim 5, wherein the AgBaG moiety further comprises at least one oxide of aluminum, sodium, or potassium.

7. The biomaterial according to claim 5, wherein the AgBaG moiety further comprises silver oxide (Ag₂O), silver ions (Ag⁺), or a combination thereof.

8. The biomaterial according to claim 1, wherein the GelMA-AgBaG is dissolved in an aqueous solvent.

9. The biomaterial according to claim 1, further comprising a photoinitiator.

10. The biomaterial according to claim 1, further comprising a plurality of cells.

11. The biomaterial according to claim 1, further comprising a plurality of cell-derived products.

12. The biomaterial according to claim 11, wherein the plurality of cell-derived products includes extracellular vesicles.

13. The biomaterial according to claim 1, further comprising an additive selected from the group consisting of growth factors, antimicrobial agents, active pharmaceutical ingredients, polynucleotides, and combinations thereof.

14. The biomaterial according to claim 1, further comprising an adjunct agent selected from the group consisting of preservatives, visualization markers, imaging agents, monitoring agents, agents for increasing efficiency, agents for increasing cytocompatibility, agents for increasing biocompatibility, agents for modulating an immune response, agents for improving function of the biomaterial, agents for providing an additional function to the biomaterial, and combinations thereof.

15. A biomaterial comprising:
a methacrylated polymer; and
a silver-containing bioactive glass (AgBaG) moiety,
wherein the AgBaG moiety is chemically attached to the methacrylated polymer to define methacrylated polymer-AgBaG.

16. A construct comprising the biomaterial according to claim 15, wherein the biomaterial is crosslinked.

17. The construct according to claim 15, wherein the construct is configured as a scaffold.

18. The construct according to claim 16, comprising at least one type of living cell or cellular product embedded within the crosslinked biomaterial.

19. A method of treating a disorder or augmenting a structure or function of a tissue in a subject in need thereof, the method comprising:
disposing the construct according to claim 16 on a location of the disorder or the tissue in the subject.

20. The method according to claim 19, wherein the disorder is a bone defect and the method comprises disposing the construct in a site of the bone defect.

21. The method according to claim 20, wherein the construct is a patient-specific construct having a three-dimensional geometry that is a negative of the bone defect.

22. The method according to claim 19, wherein the disorder is a hemorrhage and the method comprises disposing the construct at a source of the hemorrhage.

23. The method according to claim 19, wherein the disorder is an aneurysm and the method comprises disposing the construct at the site of the aneurysm.

24. The method according to claim 19, wherein the disorder is a skin defect and the method comprises disposing the construct at the site of the skin defect, wherein the skin defect is a wound, burn, pressure sore, or excision.

25. The method according to claim 19, wherein the disorder is inflamed tissue associated with osteomyelitis, periimplantitis, periodontitis, or combinations thereof and the method comprises disposing the construct at the site of the inflamed tissue.

26. A method of making the construct according to claim 16, the method comprising:
three-dimensionally printing the construct from the biomaterial; and
crosslinking the biomaterial to form the construct.

27. A method of treating a defect or augmenting a structure or function of a tissue in a subject in need thereof, the method comprising:
applying the biomaterial according to claim 1 to a target site associated with the defect or tissue and crosslinking the biomaterial in situ to form a three-dimensional construct comprising the GelMA-AgBaG at the target site.

28. A method of making a biomaterial, the method comprising:
combining a first solution comprising gelatin methacryloyl (GelMA), a linker, and a second solution comprising glass-ceramic precursors and silver (Ag) to form a biomaterial precursor solution; and
incubating the biomaterial precursor solution at greater than or equal to about 25° C. to less than or equal to about 100° C. for a time sufficient to form the biomaterial comprising GelMA-Ag-containing bioactive glass (GelMA-AgBaG),
wherein the AgBaG is chemically coupled to the GelMA by way of the linker.

29. The method according to claim 28, wherein the first solution comprises the GelMA dissolved in phosphate buffered saline (PBS).

30. The method according to claim 28, wherein the second solution comprises the glass-ceramic precursors and Ag dissolved in an aqueous solvent, wherein the glass-ceramic precursors comprise a silicon oxide (SiO₂) precursor, a calcium oxide (CaO) precursor, a phosphorus pentoxide (P₂O₅) precursor, and optionally at least one of an aluminum oxide (Al₂O₃) precursor, a sodium oxide (Na₂O) precursor, or a potassium oxide (K₂O) precursor.

31. The method according to claim 28, wherein the incubating is performed for greater than or equal to about 1 hour to less than or equal to about 24 hours.

32. The method according to claim 28, wherein the linker is (3-glycidyloxypropyl) trimethoxysilane (GPTMS).

33. The method according to claim 28, further comprising combining a photoinitiator with the first solution, the linker, and the second solution.

34. The method according to claim 28, wherein the GelMA is prepared by:
incubating gelatin type A and methacrylic acid (MAA) in a carbonate-bicarbonate (CB) buffer with a pH of 7-9 or phosphate buffered saline (PBS) with a pH of 7-9 for greater than or equal to about 0.5 hours to less than or equal to about 12 hours to form a GelMA solution;
filtering the GelMA solution to remove impurities; and
dialyzing the GelMA solution against water.

35. The method according to claim 28, wherein the GelMA is prepared by:
dissolving gelatin type A in dimethyl sulfoxide (DMSO) to form a gelatin solution;
adding methacrylic acid (MAA) to the gelatin solution over a time period of greater than or equal to about 0.5 hours to less than or equal to about 6 hours to form the GelMA in the gelatin solution;
precipitating the GelMA by adding toluene to the gelatin solution at about 2 times to 5 times the volume of the DMSO in the gelatin solution;
removing the DMSO and the toluene to isolate the GelMA; and
washing the GelMA with water.
